(19) United States
(12) Patent Application Publication
(10) Pub. No.: US 2018/0369205 A1
(43) Pub. Date: Dec. 27, 2018

(54) Heterobifunctional Pan-Selectin Antagonists Having a Triazole Linker

(71) Applicant: GlycoMimetics, Inc., Rockville, MD (US)

(72) Inventors: Beat ERNST, Magden (CH); Beatrice WAGNER, Aesch BL (CH)

(73) Assignee: GlycoMimetics, Inc., Rockville, MD (US)

(21) Appl. No.: 15/781,072

(22) PCT Filed: Nov. 30, 2016
(86) PCT No.: PCT/US2016/064189

§ 371 Date: Jun. 1, 2018

Related U.S. Application Data
(60) Provisional application No. 62/262,155, filed on Dec. 2, 2015.

(51) Int. Cl. A61K 31/4192; A61K 31/7056; A61P 25/08; A61P 35/02; A61P 7/00; A61P 37/06; C07H 15/26

(52) U.S. Cl. CPC A61K 31/4192; A61K 31/7056; A61P 25/08; C07H 15/26; A61P 7/00; A61P 37/06; A61P 35/02

(57) ABSTRACT

Compounds, compositions, and methods for modulating in vitro and in vivo processes mediated by selectin binding. For example, heterobifunctional compounds that inhibit both E-selectins and P-selectins are described, wherein the selectin modulators that modulate (e.g., inhibit or enhance) a selectin-mediated function comprise particular glycomimetics linked to a member of a class of compounds termed BASAs (Benzyl Amino Sulfonic Acids). The compounds are of formula (Ia) wherein the substituents are as defined in the claims.

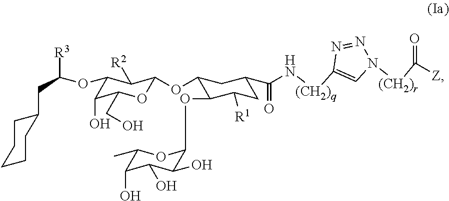

### RELATED APPLICATIONS

This application claims the benefit of and priority from U.S. Provisional Patent Application 62/262,155, filed Dec. 2, 2015. The foregoing application is incorporated herein by reference in its entirety.

### FIELD OF INVENTION

Compounds, compositions, and methods for modulating in vitro and in vivo processes mediated by selectin binding are described herein. For example, selectin modulators and their use are described, wherein the selectin modulators comprise a glycomimetic linked via a triazole linker to a member of a class of compounds termed BASAs (Benzyl Amino Sulfonic Acids).

### BACKGROUND OF THE INVENTION

When a tissue is infected or damaged, the inflammatory process directs leukocytes and other immune system components to the site of infection or injury. Within this process, leukocytes play a role in the engulfment and digestion of microorganisms. Thus, the recruitment of leukocytes to infected or damaged tissue is important for mounting an effective immune defense.

Selectins are a group of structurally similar cell surface receptors that are important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region, and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells and binds to the carbohydrate sialyl-Lewis^{x} (SLe^{x}) which is presented as a glycoprotein or glycolipid on the surface of certain leukocytes (monocytes and neutrophils) and helps these cells adhere to capillary walls in areas where surrounding tissue is infected or damaged. E-selectin also binds to sialyl-Lewis^{a} (SLe^{a}) which is expressed on many tumor cells. P-selectin is expressed on inflamed endothelium and platelets and also recognizes SLe^{x} and SLe^{a} but also contains a second site that interacts with sulfated tyrosine. The expression of E-selectin and P-selectin is generally increased when the tissue adjacent to a capillary which is infected or damaged. L-selectin is expressed on leukocytes. Selectin-mediated intercellular adhesion and formation of new capillaries during angiogenesis are examples of selectin-mediated functions.

Modulators of selectin-mediated function include the PSGL-1 protein (and smaller peptide fragments thereof), fucoidan, glycyrrhizin (and derivatives), anti-selectin antibodies, sulfated lactose derivatives, heparin and heparin fragments, sulfated hyaluronic acid, condroitin sulfate, sulfated dextran, sulfatides, and particular glycomimetic compounds (see, e.g., U.S. RE44,778). All but the glycomimetics have shown to be unsuitable for drug development due to insufficient activity, toxicity, lack of specificity, poor ADME characteristics, and/or availability of material.

Although selectin-mediated cell adhesion is required for fighting infection and destroying foreign material, there are situations in which cell adhesion is undesirable or excessive resulting in tissue damage instead of repair. For example, pathologies that involve abnormal adhesion of white blood cells include autoimmune and inflammatory diseases, shock, and reperfusion injuries. Abnormal cell adhesion may also play a role in transplant and graft rejection. In addition, some circulating cancer cells appear to take advantage of the inflammatory mechanism to bind to activated endothelium. In these circumstances, modulation of selectin-mediated intercellular adhesion may be desirable.

Accordingly, there is a need in the art for identifying modulators, e.g., inhibitors, of selectin-mediated function, e.g., of selectin-dependent cell adhesion, and for the development of methods employing such compounds to inhibit conditions associated with excessive selectin activity. The present disclosure may fulfill one or more of these needs and/or may provide other advantages.

### SUMMARY OF THE INVENTION

Briefly stated, compounds, compositions, and methods for modulating selectin-mediated processes are disclosed. The compounds comprise a glycomimetic linked via a triazole linker to a member of a class of compounds termed BASAs. The compounds may be combined with at least one additional pharmaceutically acceptable ingredient to form a pharmaceutical composition. The compounds or compositions may be used in a method to modulate (e.g., inhibit or enhance) a selectin-mediated function, such as inhibiting a selectin-mediated intercellular adhesion.

In some embodiments, selectin-modulators of Formula (I):

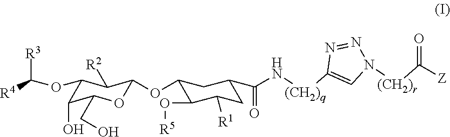

prodrugs thereof, and pharmaceutically acceptable salts of any of the foregoing are disclosed, wherein

R¹ is chosen from H, C₁₋₈ alkyl, C₂₋₈ alkenyl, C₂₋₈ alkynyl, C₄₋₁₆ cycloalkylalkyl

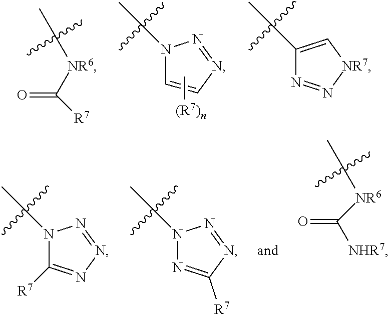

groups;

R² is chosen from C₁₋₈ alkyl, C₂₋₈ alkenyl, C₂₋₈ alkynyl, C₄₋₁₆ cycloalkylalkyl, —OH, —OX¹, halo, —NH₂, —OC(═O)X′, —NHC(═O)X¹, and —NHC(═O)NHX¹ groups, wherein X¹ is chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups;

R³ is chosen from —CN, —CH₂CN, and —C(═O)X² groups, wherein X² is chosen from C₁₋₈ alkyl, C₂₋₈ alkenyl, C₂₋₈ alkynyl, —OY², —NHOH, —NHOCH₃, —NHCN, and —NY²Y³ groups, wherein Y² and Y³, which may be identical or different, are independently chosen from H, C₁₋₈ alkyl, C₂₋₈ alkenyl, C₂₋₈ alkynyl, and C₄₋₁₆ cycloalkylalkyl groups, wherein Y² and Y³ may join together to form a ring;

R⁴ is chosen from H, C₁₋₈ alkyl, C₂₋₈ alkenyl, C₂₋₈ alkynyl, C₄₋₁₆ cycloalkylalkyl, and C₆₋₁₈ aryl groups;

R⁵ is chosen from H, mannose, arabinose, galactose, polyols,

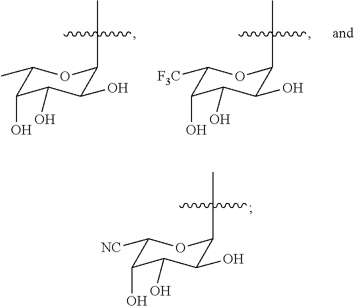 [SMILES: *C1OC(C)C(O)C(O)C1O ; *C1OC(C(F)(F)F)C(O)C(O)C1O ; *C1OC(C#N)C(O)C(O)C1O]

R⁶ is chosen from H, C₁₋₈ alkyl, C₂₋₈ alkenyl, C₂₋₈ alkynyl, C₄₋₁₆ cycloalkylalkyl, and —C(═O)R⁷ groups;

each R⁷ is independently chosen from H, C₁₋₈ alkyl, C₂₋₈ alkenyl, C₂₋₈ alkynyl, C₄₋₁₆ cycloalkylalkyl,

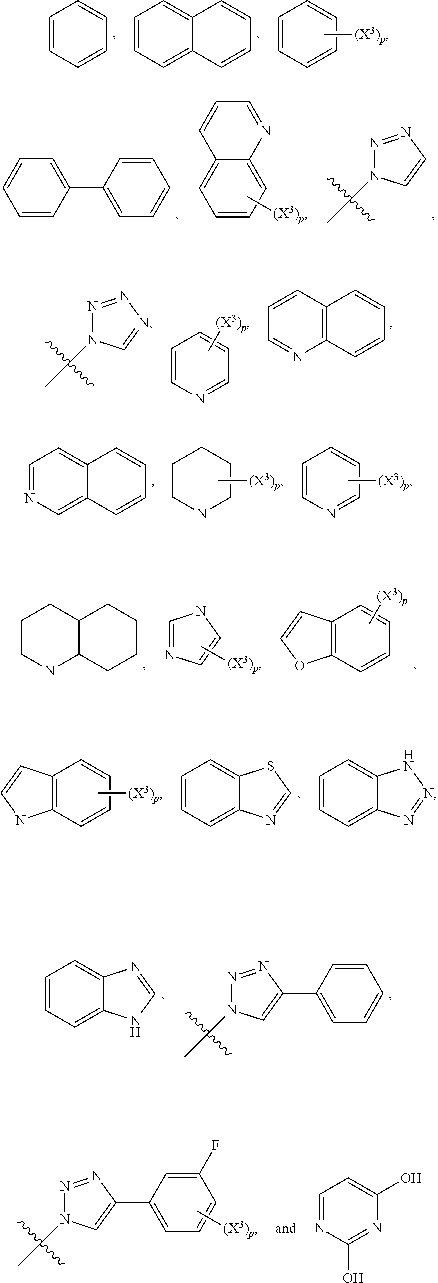

groups, wherein each X³ is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein any of the above ring compounds may be substituted with one to three groups independently chosen from Cl, F, C₁₋₈ alkyl, C₂₋₈ alkenyl, C₂₋₈ alkynyl, C₆₋₁₄ aryl, and —OY⁴ groups, wherein Y⁴ is chosen from H, C₁₋₈ alkyl, C₂₋₈ alkenyl, C₂₋₈ alkynyl, and C₆₋₁₄ aryl groups;

n is chosen from integers ranging from 0 to 2;

p is chosen from integers ranging from 0 to 3;

q is chosen from integers ranging from 1 to 10;

r is chosen from integers ranging from 1 to 10; and

Z is chosen from BASA groups.

As used herein, “compound of Formula (I)” includes selectin-modulators of Formula (I), pharmaceutically acceptable salts of selectin-modulators of Formula (I), prodrugs of selectin-modulators of Formula (I), and pharmaceutically acceptable salts of prodrugs of selectin-modulators of Formula (I).

In some embodiments, pharmaceutical compositions comprising at least one compound of Formula (I) and optionally at least one additional pharmaceutically acceptable ingredient are presented.

In some embodiments, a compound of Formula (I) and/or a pharmaceutical composition comprising at least one compound of Formula (I) may be used for the preparation and/or manufacture of a medicament for use in treating at least one of the diseases, disorders, and conditions described herein.

In some embodiments, a method for modulating a selectin-mediated function comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. In some embodiments, the selectin-mediated function is enhanced. In some embodiments, the selectin-mediated function is inhibited.

In some embodiments, a method for contacting a cell expressing a selectin to modulate (e.g., stimulate or inhibit) the selectin's function comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for inhibiting the development of a condition associated with an excessive selectin-mediated function comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. In some embodiments, the selectin-mediated function is selectin-mediated intercellular adhesion.

In some embodiments, a method for inhibiting rejection of a transplanted tissue comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for treating sickle cell disease (SCD) or complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. In some embodiments, this application provides a method for treating vaso-occlusive crisis comprising administering to a subject in need thereof an effective amount of at least one compound of Formula (I) and/or a pharmaceutical composition comprising at least one compound of Formula (I) and optionally at least one additional pharmaceutically acceptable ingredient.

In some embodiments, a method for treating graft versus host disease (GVHD) or complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for treating sinusoidal obstruction syndrome (SOS) or complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In some embodiments, a method for treating cancers of the blood and complications associated therewith comprising administering to a subject in need thereof an effective amount of at least one compound of Formula (I) and/or a pharmaceutical composition comprising at least one compound of Formula (I) and optionally at least one additional pharmaceutically acceptable ingredient is disclosed. Examples of cancers of the blood include, but are not limited to, acute myelogenous leukemia (AML), acute lymphoblastic leukemia (ALL), chronic myelogenous leukemia (CIVIL) and multiple myeloma (MM).

In some embodiments, a method for treating epilepsy comprising administering to a subject in need thereof an effective amount of at least one compound of Formula (I) and/or a pharmaceutical composition comprising at least one compound of Formula (I) and optionally at least one additional pharmaceutically acceptable ingredient is disclosed.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description.

### DETAILED DESCRIPTION

Disclosed herein are compounds, compositions, and methods for modulating selectin-mediated functions. The compounds have a variety of uses in vitro and in vivo.

In some embodiments, presented are selectin-modulators of Formula (I):

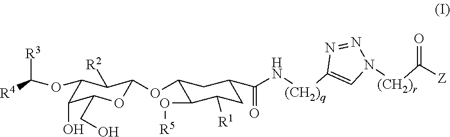

prodrugs thereof, and pharmaceutically acceptable salts of any of the foregoing are disclosed, wherein

R¹ is chosen from H, C₁₋₈ alkyl, C₂₋₈ alkenyl, C₂₋₈ alkynyl, C₄₋₁₆ cycloalkylalkyl

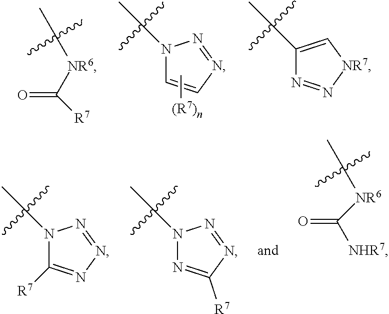

groups;

R² is chosen from C₁₋₈ alkyl, C₂₋₈ alkenyl, C₂₋₈ alkynyl, C₄₋₁₆ cycloalkylalkyl, —OH, —OX¹, halo, —NH₂, —OC(═O)X′, —NHC(═O)X¹, and —NHC(═O)NHX¹ groups, wherein X¹ is chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups;

R³ is chosen from —CN, —CH₂CN, and —C(═O)X² groups, wherein X² is chosen from C₁₋₈ alkyl, C₂₋₈ alkenyl, C₂₋₈ alkynyl, —OY², —NHOH, —NHOCH₃, —NHCN, and —NY²Y³ groups, wherein Y² and Y³, which may be identical or different, are independently chosen from H, C₁₋₈ alkyl, C₂₋₈ alkenyl, C₂₋₈ alkynyl, and C₄₋₁₆ cycloalkylalkyl groups, wherein Y² and Y³ may join together to form a ring;

R⁴ is chosen from H, C₁₋₈ alkyl, C₂₋₈ alkenyl, C₂₋₈ alkynyl. C₄₋₁₆ cycloalkylalkyl, and C₆₋₁₈ aryl groups;

R⁵ is chosen from H, mannose, arabinose, galactose, polyols,

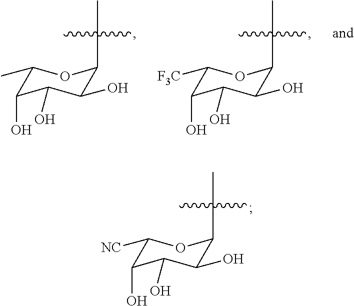 [SMILES: *C1OC(C)C(O)C(O)C1O ; *C1OC(C(F)(F)F)C(O)C(O)C1O ; *C1OC(C#N)C(O)C(O)C1O]

R⁶ is chosen from H, C₁₋₈ alkyl, C₂₋₈ alkenyl, C₂₋₈ alkynyl, C₄₋₁₆ cycloalkylalkyl, and —C(═O)R⁷ groups;

each R⁷ is independently chosen from H, C₁₋₈ alkyl, C₂₋₈ alkenyl, C₂₋₈ alkynyl, C₄₋₁₆ cycloalkylalkyl,

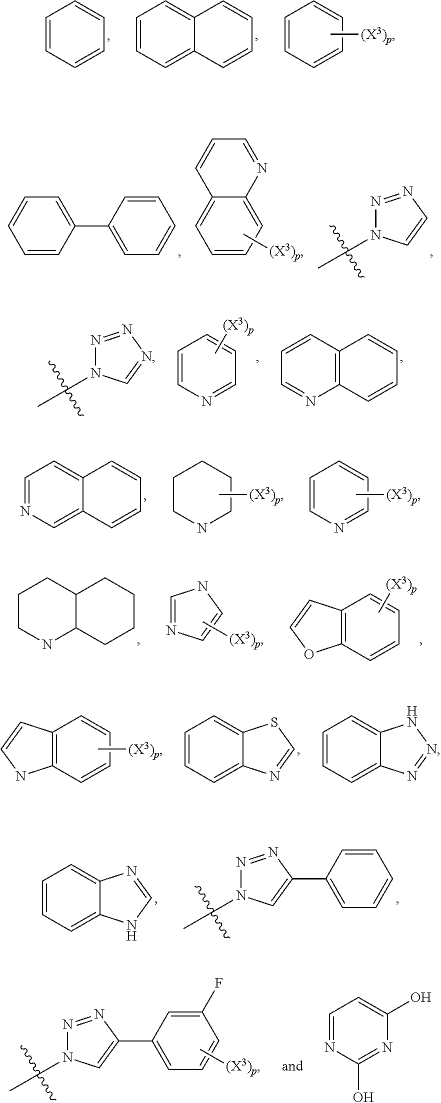

groups, wherein each X³ is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein any of the above ring compounds may be substituted with one to three groups independently chosen from Cl, F, C₁₋₈ alkyl, C₂₋₈ alkenyl, C₂₋₈ alkynyl, C₆₋₁₄ aryl, and —OY⁴ groups, wherein Y⁴ is chosen from H, C₁₋₈ alkyl, C₂₋₈ alkenyl, C₂₋₈ alkynyl, and C₆₋₁₄ aryl groups;

n is chosen from integers ranging from 0 to 2;

p is chosen from integers ranging from 0 to 3;

q is chosen from integers ranging from 1 to 10;

r is chosen from integers ranging from 1 to 10; and

Z is chosen from BASA groups.

BASAs (Benzyl Amino Sulfonic Acids) are low molecular weight sulfated compounds which have the ability to interact with a selectin. The interaction modulates or assists in the modulation (e.g., inhibition or enhancement) of a selectin-mediated function (e.g., an intercellular interaction). They exist as either their protonated acid form, or as a sodium salt, although sodium may be replaced with potassium or any other pharmaceutically acceptable counterion.

Portions of BASA that retain the ability to interact with a selectin (which interaction modulates or assists in the modulation of a selectin-mediated function as described herein) are also a “BASA” of the disclosed selectin modulators. Such portions generally comprise at least one aromatic ring present within the BASA structure. In some embodiments, a portion may comprise a single aromatic ring, multiple such rings, or half of a symmetrical BASA molecule.

Analogues of BASA and portions thereof (both of which possess the biological characteristic set forth herein) are also encompassed, e.g., by the “BASA” group of the selectin modulators within the disclosure. As used herein, an “analogue” is a compound that differs from BASA or a portion thereof because of one or more additions, deletions and/or substitutions of chemical moieties, such that the ability of the analogue to inhibit a selectin-mediated interaction is not diminished. For example, an analogue may contain S to P substitutions (e.g., a sulfate group replaced with a phosphate group). Other possible modifications include: (a) modifications to ring size (e.g., any ring may contain between 4 and 7 carbon atoms); (b) variations in the number of fused rings (e.g., a single ring may be replaced with a polycyclic moiety containing up to three fused rings, a polycyclic moiety may be replaced with a single unfused ring or the number of fused rings within a polycyclic moiety may be altered); (c) ring substitutions in which hydrogen atoms or other moieties covalently bonded to a carbon atom within an aromatic ring may be replaced with any of a variety of moieties, such as F, Cl, Br, I, OH, OC₁₋₈ alkyl, SH, NO₂, CN, NH₂, NHC₁₋₈ alkyl, N(C₁₋₈ alkyl)₂, SO₃T (wherein T is chosen from H⁺, Na⁺, K⁺, and other pharmaceutically acceptable counterions), —CO₂T, —PO₄T₂, —SO₂NH₂, C₁₋₈ alkyl, C₆₋₁₀ aryl, —C(═O)OC₁₋₈ alkyl, —CF₂Q (wherein Q is chosen from H, F, alkyl, aryl, and acyl groups) and carbohydrates; and (d) modifications to linking moieties (i.e., moieties located between rings in the BASA molecule) in which groups such as alkyl, ester, amide, anhydride, and carbamate groups may be substituted for one another.

In some embodiments, R¹ is chosen from H, C₁₋₈ alkyl, C₂₋₈ alkenyl, C₂₋₈ alkynyl, and

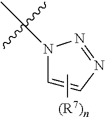

groups. In some embodiments, R¹ is chosen from H and C₁₋₈ alkyl groups. In some embodiments, R¹ is chosen from C₁₋₈ alkyl groups. In some embodiments, R¹ is chosen from C₁₋₄ alkyl groups. In some embodiments, R¹ is methyl. In some embodiments, R¹ is ethyl.

In some embodiments, R¹ is chosen from methyl, ethyl, and

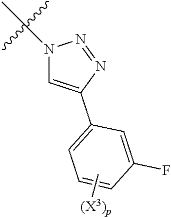

groups.

In some embodiments, R is

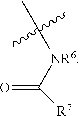

In some embodiments, R¹ is

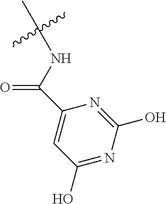 [SMILES: *NC(=O)c1cc(O)nc(O)n1]

In some embodiments, R² is chosen from —OC(═O)X′, —NHC(═O)X¹, and —NHC(═O)NHX¹ groups, wherein X¹ is chosen from C₁₋₈ alkyl, C₂₋₁₂ heterocyclyl, C₆₋₁₈ aryl, and C₁₋₁₃ heteroaryl groups. In some embodiments, R² is chosen from

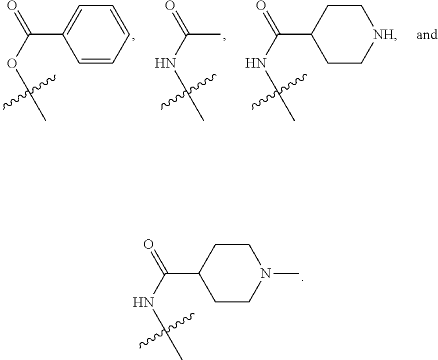

In some embodiments, R³ is chosen from —C(═O)X² groups, wherein X² is chosen from —OY², —NHOH, —NHOCH₃, and —NY²Y³ groups, wherein Y² and Y³, which may be identical or different, are independently chosen from H and C₁₋₈ alkyl, wherein Y² and Y³ may joint together to form a ring. In some embodiments, R³ is —C(═O)OH.

In some embodiments, R⁴ is chosen from C₄₋₁₆ cycloalkylalkyl groups. In some embodiments, R⁴ is chosen from C₄₋₈ cycloalkylalkyl groups. In some embodiments, R⁴ is chosen from cyclopropylmethyl and cyclohexylmethyl. In some embodiments, R⁴ is cyclopropylmethyl. In some embodiments R⁴ is cyclohexylmethyl.

In some embodiments, R⁵ is chosen from

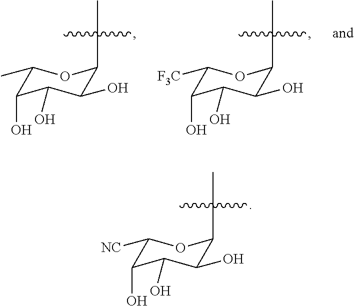 [SMILES: *C1OC(C)C(O)C(O)C1O ; *C1OC(C(F)(F)F)C(O)C(O)C1O ; *C1OC(C#N)C(O)C(O)C1O]

In some embodiments, R⁵ is fucose.

In some embodiments, R⁶ is chosen from H, C₁₋₈ alkyl, and —C(═O)R⁷ groups. In some embodiments, R⁶ is chosen from H and C₁₋₈ alkyl groups. In some embodiments, R⁶ is chosen from C₁₋₄ alkyl groups. In some embodiments, R⁶ is H.

In some embodiments, each R⁷ independently chosen from H, C₁₋₈ alkyl, and

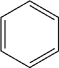

groups, wherein any of the above ring compounds may be substituted with one to three groups independently chosen from Cl, F, C₁₋₈ alkyl, C₆₋₁₄ aryl, and —OY⁴ groups, wherein each Y⁴ is independently chosen from H, C₁₋₈ alkyl, and C₆₋₁₄ aryl groups. In some embodiments, at least one R⁷ is independently chosen from C₁₋₈ alkyl groups. In some embodiments, at least one R⁷ is chosen from C₁₋₄ alkyl groups. In some embodiments, at least one R⁷ is chosen from methyl and ethyl. In some embodiments, at least one R⁷ is H. In some embodiments, at least one R⁷ is methyl. In some embodiments, at least one R⁷ is ethyl.

In some embodiments, each R⁷ is independently chosen from H, C₁₋₈ alkyl,

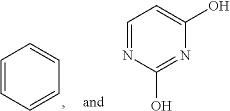 [SMILES: c1ccccc1 ; Oc1ccnc(O)n1]

groups, wherein any of the above ring compounds may be substituted with one to three groups independently chosen from Cl, F, C₁₋₈ alkyl, C₆₋₁₄ aryl, and —OY⁴ groups, wherein each Y⁴ is independently chosen from H, C₁₋₈ alkyl, and C₆₋₁₄ aryl groups.

In some embodiments, q is chosen from integers ranging from 2 to 4. In some embodiments, q is 2. In some embodiments, q is 3. In some embodiments, q is 4.

In some embodiments, r is chosen from integers ranging from 2 to 6. In some embodiments, r is 2. In some embodiments, r is 3. In some embodiments, r is 4.

In some embodiments, n is 0. In some embodiments, n is 1. In some embodiments, p is 0. In some embodiments, p is 1.

In some embodiments, Z comprises a compound chosen from

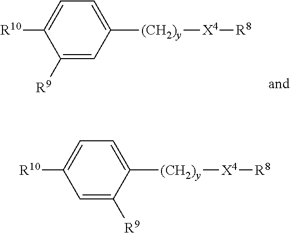

wherein

R⁸ is chosen from H, —F, and —C(═O)OY⁵ groups;

R⁹ is chosen from H, —PO₃T₂, —SO₃T₂, —CH₂—PO₃T₂, —CH₂—SO₃T₂, —CF₃, —CHR¹¹R¹², —CH₂CHR¹¹R¹², —(CH₂)₂CHR¹¹R¹², —(CH₂)₃CHR¹¹R¹², and —NZ³Z⁴ groups, wherein R¹¹ and R¹², which may be identical or different, are independently chosen from H, —C(═O)OY⁵, and —NH₂ groups, wherein Z³ and Z⁴, which may be identical or different, are independently chosen from H and —Y⁶—(C₆₋₁₈ aryl) groups, wherein Y⁶ is chosen from C₁₋₄ alkyl and C(═O) groups;

R¹⁰ is chosen from H, —PO₃T₂, —SO₃T₂, —CH₂—PO₃T₂, —CH₂—SO₃T₂, —CF₃, —CHR¹³R¹⁴, —CH₂CHR¹³R¹⁴, —(CH₂)₂CHR¹³R¹⁴, —(CH₂)₃CHR¹³R¹⁴, and —NZ⁵Z⁶ groups, wherein R¹³ and R¹⁴, which may be identical or different, are independently chosen from H, —C(═O)OY⁵, —NH₂ groups, wherein Z⁵ and Z⁶, which may be identical or different, are independently chosen from H and —Y⁷—(C₆₋₁₈ aryl) groups, wherein Y⁷ is chosen from C₁₋₄ alkyl and C(═O) groups;

X⁴ is chosen from —PO₂T-, —SO₂T-, and —CF₂—;

each Y⁵ is independently chosen from H, C₁₋₄ alkyl, and C₆₋₁₈ aryl groups;

each T is independently chosen from H and pharmaceutically acceptable counterions; and

y is chosen from integers ranging from 0 to 1.

In some embodiments, Z is chosen from

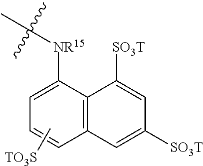

wherein

R¹⁵ is chosen from H, C₁₋₈ alkyl, —C(═O)X⁵, and —C(═O)NHX⁵ groups, wherein X⁵ is chosen from C₁₋₈ alkyl, C₂₋₈ alkenyl, C₂₋₈ alkynyl, C₆₋₁₈ aryl, and C₁₋₁₃ heteroaryl groups; and

each T is independently chosen from H and pharmaceutically acceptable counterions.

In some embodiments, Z is chosen from

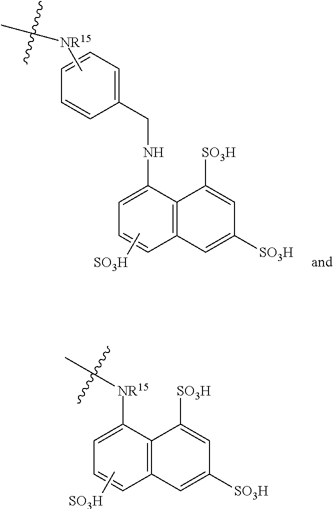

wherein

R¹⁵ is chosen from H, C₁₋₈ alkyl, —C(═O)X⁵, and —C(═O)NHX⁵ groups, wherein X⁵ is chosen from H, C₁₋₈ alkyl, C₂₋₈ alkenyl, C₂₋₈ alkynyl, C₆₋₁₈ aryl, and C₁₋₁₃ heteroaryl groups.

In some embodiments, Z is chosen from

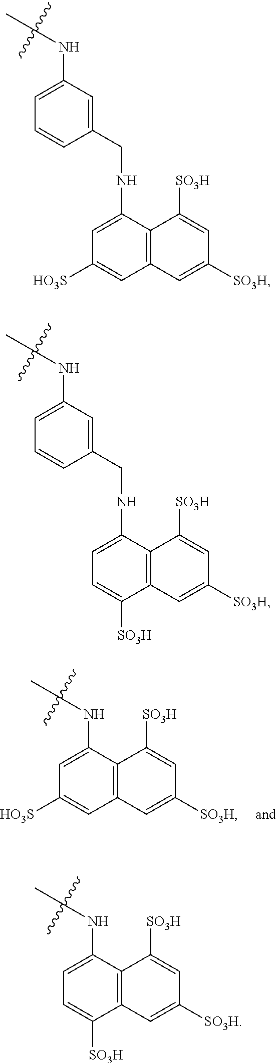 [SMILES: *Nc1cccc(CNc2cc(S(=O)(=O)O)cc3cc(S(=O)(=O)O)cc(S(=O)(=O)O)c23)c1 ; *Nc1cccc(CNc2ccc(S(=O)(=O)O)c3cc(S(=O)(=O)O)cc(S(=O)(=O)O)c23)c1 ; *Nc1cc(S(=O)(=O)O)cc2cc(S(=O)(=O)O)cc(S(=O)(=O)O)c12 ; *Nc1ccc(S(=O)(=O)O)c2cc(S(=O)(=O)O)cc(S(=O)(=O)O)c12]

In some embodiments, the selectin-modulator of Formula (I) is chosen from compounds of Formula (Ia):

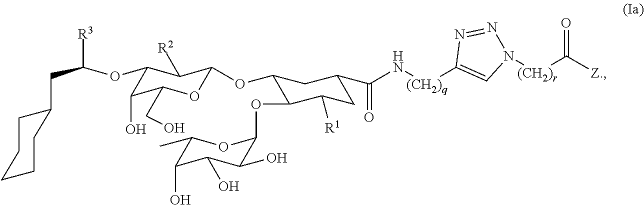

prodrugs thereof, and pharmaceutically acceptable salts of any of the foregoing.

In some embodiments, the selectin-modulator is chosen from compounds of the following Formulae:

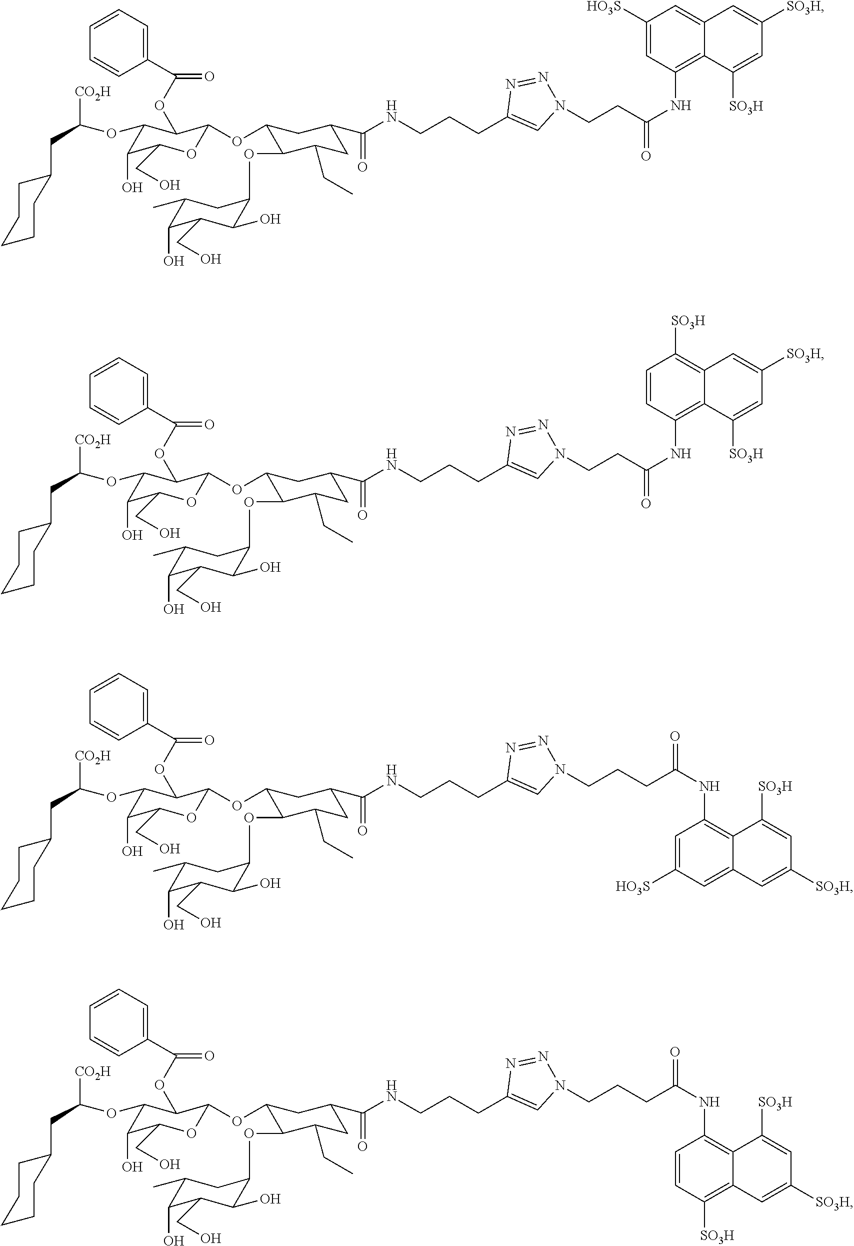

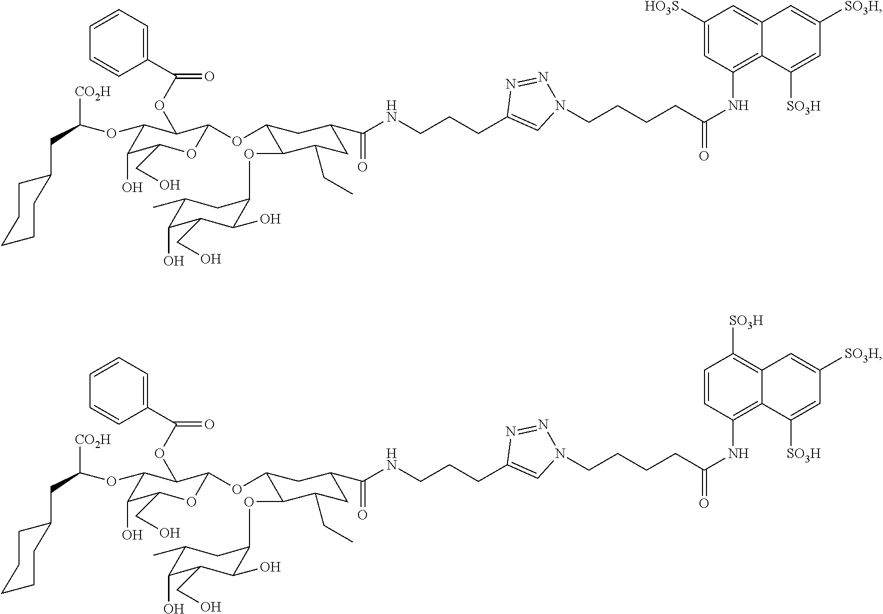 [SMILES: CCC1CC(C(=O)NCCCc2cn(CCCCC(=O)Nc3cc(S(=O)(=O)O)cc4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1CC(C)C(O)C(CO)C1O ; CCC1CC(C(=O)NCCCc2cn(CCCCC(=O)Nc3ccc(S(=O)(=O)O)c4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1CC(C)C(O)C(CO)C1O]

and prodrugs thereof, and pharmaceutically acceptable salts of any of the foregoing.

In some embodiments, the selectin modulator is chosen from compounds of the following Formulae:

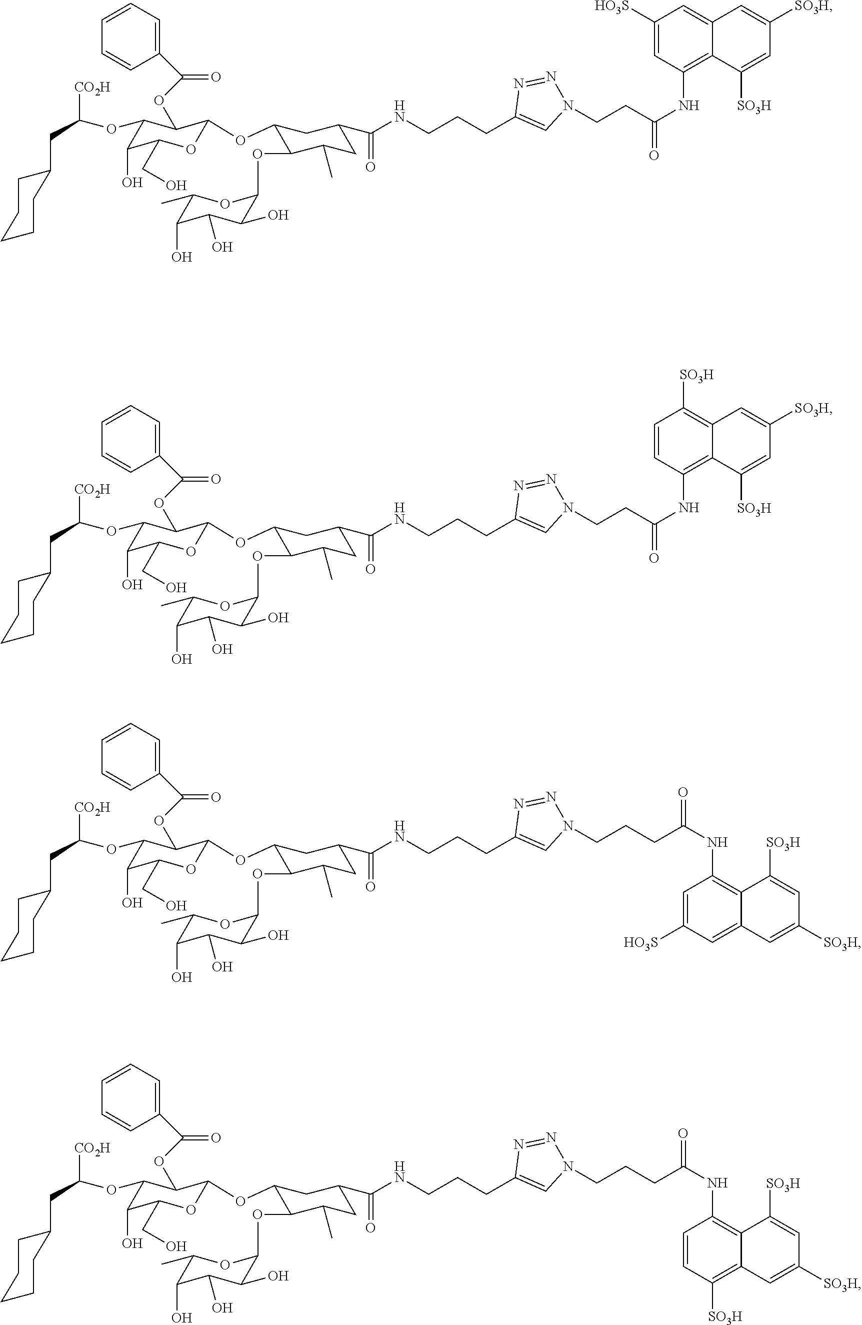

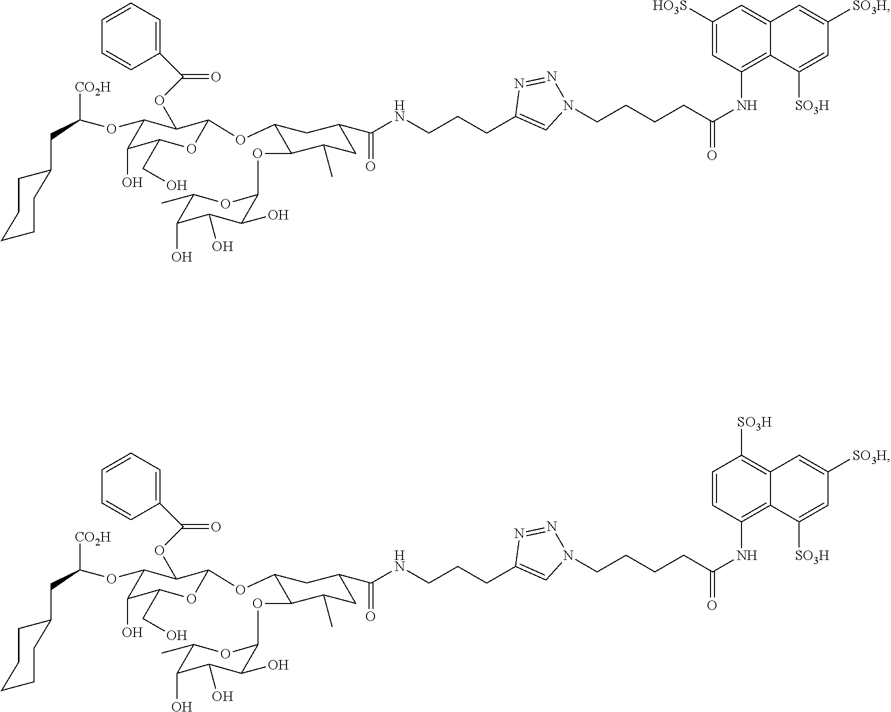 [SMILES: CC1CC(C(=O)NCCCc2cn(CCCCC(=O)Nc3cc(S(=O)(=O)O)cc4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1OC(C)C(O)C(O)C1O ; CC1CC(C(=O)NCCCc2cn(CCCCC(=O)Nc3ccc(S(=O)(=O)O)c4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1OC(C)C(O)C(O)C1O]

and prodrugs thereof, and pharmaceutically acceptable salts of any of the foregoing.

In some embodiments, the selectin modulator is chosen from compounds of the following Formulae:

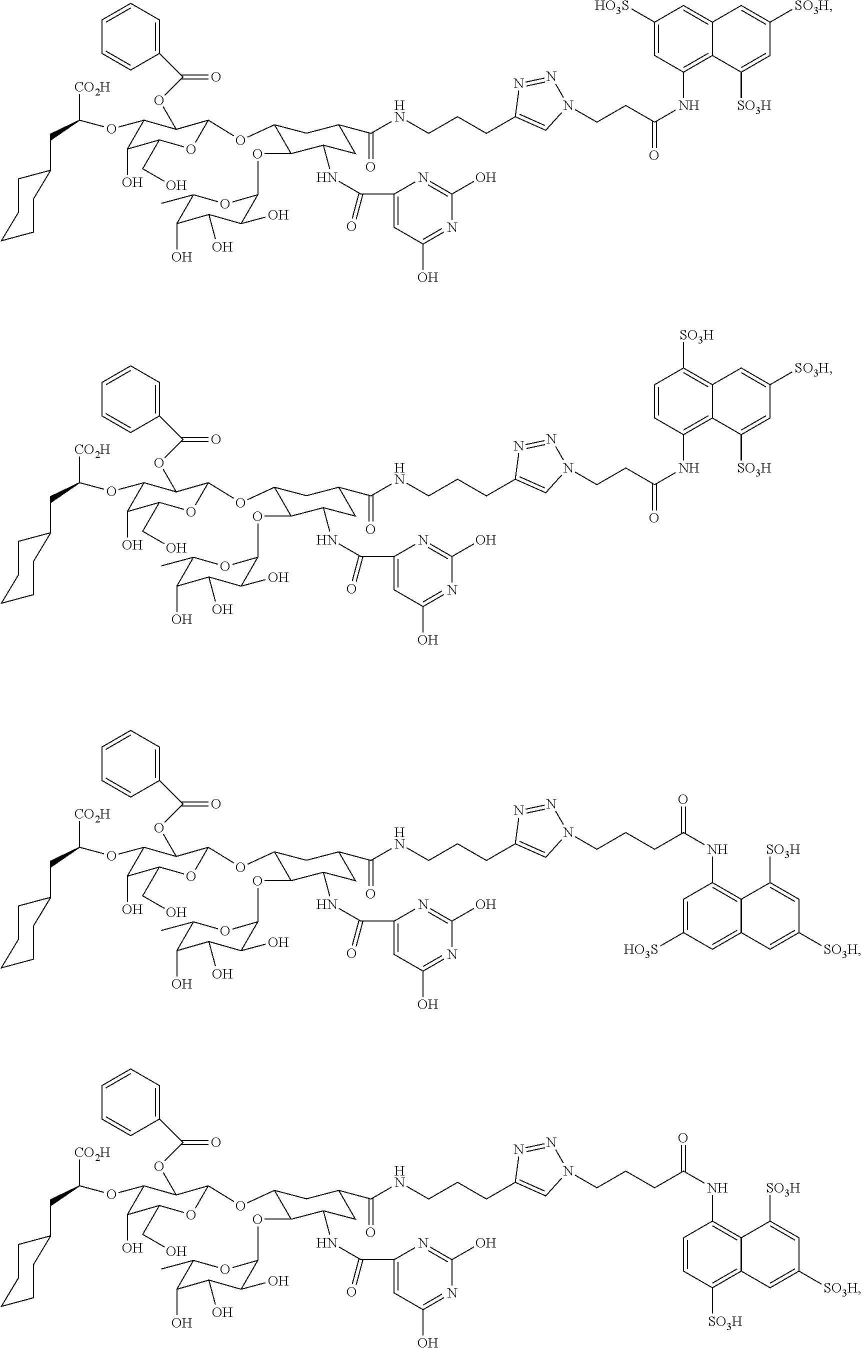

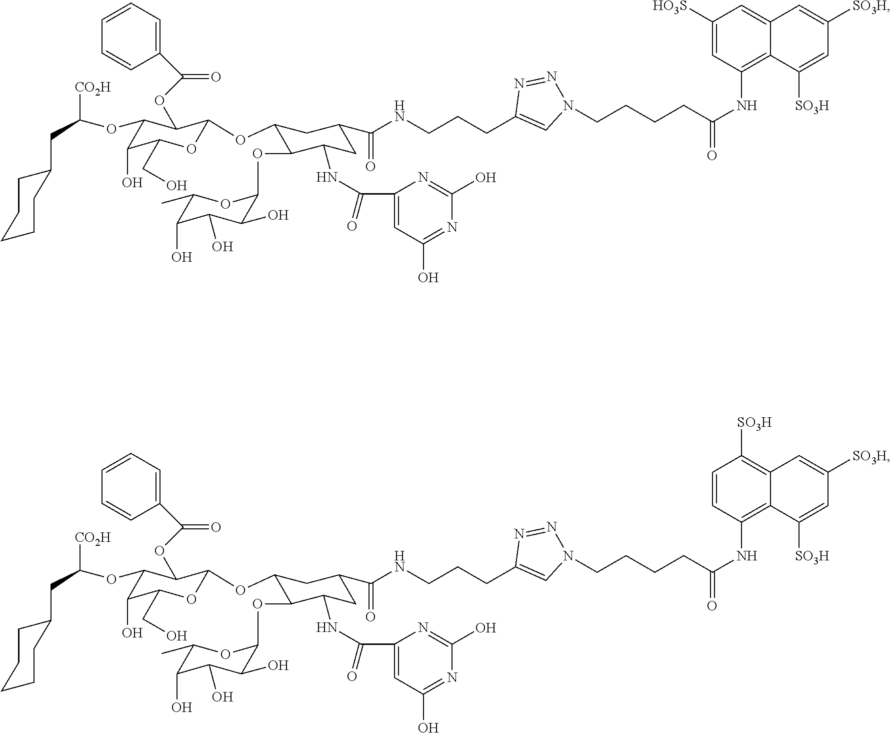 [SMILES: CC1OC(OC2C(NC(=O)c3cc(O)nc(O)n3)CC(C(=O)NCCCc3cn(CCCCC(=O)Nc4cc(S(=O)(=O)O)cc5cc(S(=O)(=O)O)cc(S(=O)(=O)O)c45)nn3)CC2OC2OC(CO)C(O)C(O[C@@H](CC3CCCCC3)C(=O)O)C2OC(=O)c2ccccc2)C(O)C(O)C1O ; CC1OC(OC2C(NC(=O)c3cc(O)nc(O)n3)CC(C(=O)NCCCc3cn(CCCCC(=O)Nc4ccc(S(=O)(=O)O)c5cc(S(=O)(=O)O)cc(S(=O)(=O)O)c45)nn3)CC2OC2OC(CO)C(O)C(O[C@@H](CC3CCCCC3)C(=O)O)C2OC(=O)c2ccccc2)C(O)C(O)C1O]

and prodrugs thereof, and pharmaceutically acceptable salts of any of the foregoing.

In some embodiments, the selectin modulator is chosen from compounds of the following Formulae:

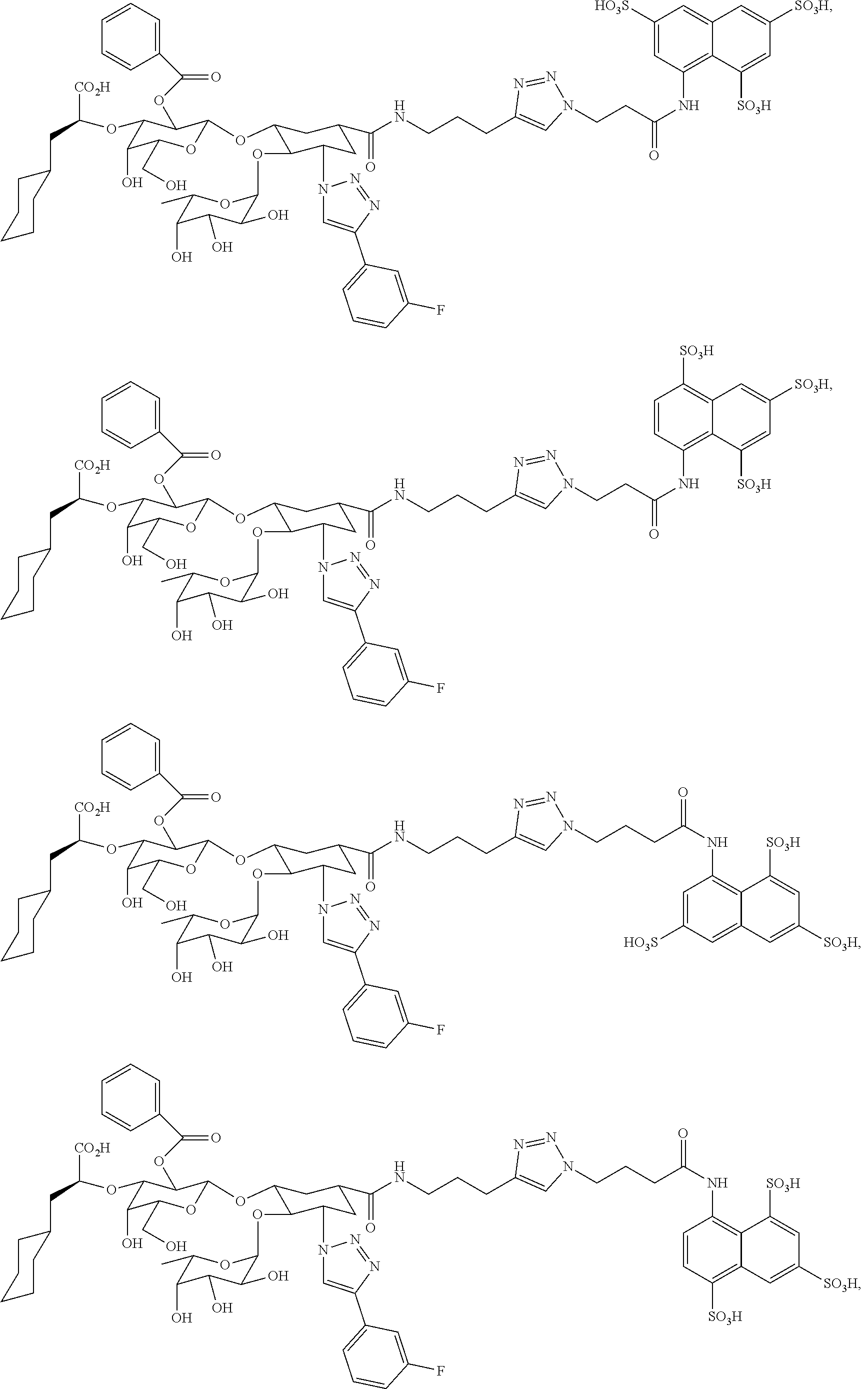

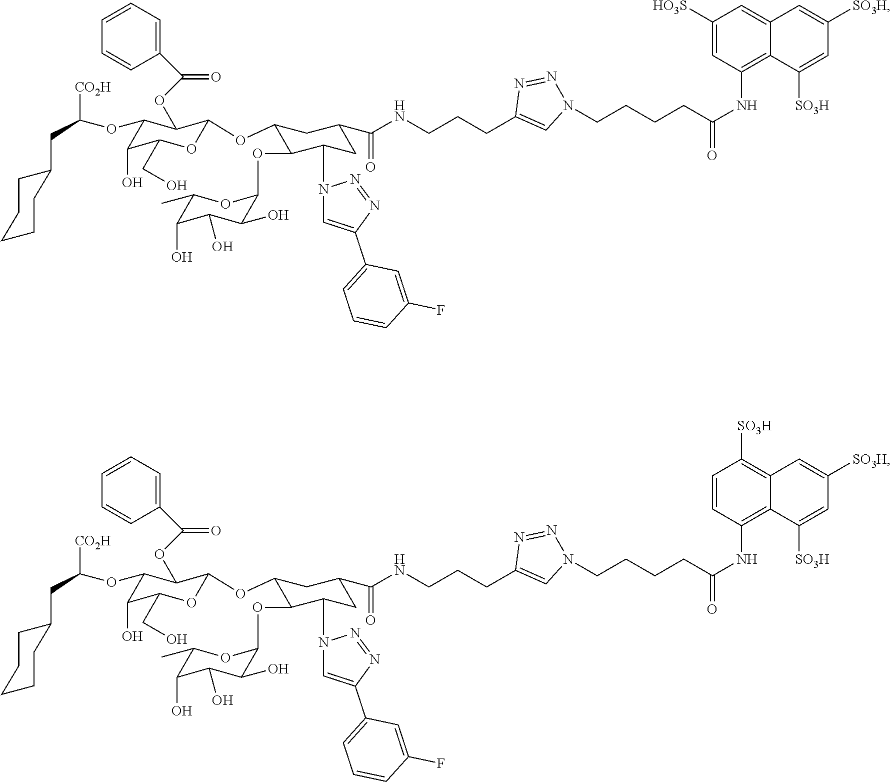 [SMILES: CC1OC(OC2C(OC3OC(CO)C(O)C(O[C@@H](CC4CCCCC4)C(=O)O)C3OC(=O)c3ccccc3)CC(C(=O)NCCCc3cn(CCCCC(=O)Nc4cc(S(=O)(=O)O)cc5cc(S(=O)(=O)O)cc(S(=O)(=O)O)c45)nn3)CC2n2cc(-c3cccc(F)c3)nn2)C(O)C(O)C1O ; CC1OC(OC2C(OC3OC(CO)C(O)C(O[C@@H](CC4CCCCC4)C(=O)O)C3OC(=O)c3ccccc3)CC(C(=O)NCCCc3cn(CCCCC(=O)Nc4ccc(S(=O)(=O)O)c5cc(S(=O)(=O)O)cc(S(=O)(=O)O)c45)nn3)CC2n2cc(-c3cccc(F)c3)nn2)C(O)C(O)C1O]

and prodrugs thereof, and pharmaceutically acceptable salts of any of the foregoing.
Also provided are pharmaceutical compositions comprising at least one compound of Formula (I) as described herein. Such pharmaceutical compositions are described in greater detail herein. These compounds and compositions may be used in the methods described herein.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present invention are generally performed, unless otherwise indicated, according to conventional methods well known in the art and as described in various general and more specific references that are cited and discussed throughout this specification.

Chemistry terms used herein are used according to conventional usage in the art, as exemplified by “The McGraw-Hill Dictionary of Chemical Terms,” Parker S., Ed., McGraw-Hill, San Francisco, Calif. (1985).

All of the publications, patents and published patent applications referred to in this application are specifically incorporated by reference herein.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to”. “Including” and “including but not limited to” are used interchangeably.

Whenever a term in the specification is identified as a range (e.g., C₁₋₄ alkyl), the range independently discloses and includes each element of the range. As a non-limiting example, C₁₋₄ alkyls includes, independently, C₁ alkyls, C₂ alkyls, C₃ alkyls, and C₄ alkyls.

The term “at least one” refers to one or more, such as one, two, etc. For example, the term “at least one C₁₋₄ alkyl” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “cycloalkyl” includes saturated monocyclic or polycyclic hydrocarbon group, which may include fused or bridged ring systems. Non-limiting examples of cycloalkyl groups include cyclopropyl, cyclobutyl, cyclopentyl, cyclohexyl, cycloheptyl, cyclooctyl, adamantyl, and norbornyl. Unless otherwise stated specifically in the specification, a cycloalkyl group may be optionally substituted.

The term “cycloalkylalkyl” includes cycloalkyl groups, as described herein, appended to the parent molecular moiety through an alkyl group, as defined herein. Non-limiting examples of cycloalkylalkyl groups include cyclopropylmethyl and cyclohexylmethyl. Unless stated otherwise specifically in the specification, a cycloalkylalkyl group may be optionally substituted.

The term “aryl” includes hydrocarbon ring system group comprising 6 to 18 carbon ring atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “fused” includes any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

The term “halo” or “halogen” includes fluoro, chloro, bromo, and iodo.

The term “heterocyclyl” or “heterocyclic ring” includes 3- to 18-membered saturated or partially unsaturated non-aromatic ring groups comprising 2 to 12 ring carbon atoms and 1 to 6 ring heteroatom(s) each independently chosen from N, O, and S. Unless stated otherwise specifically in the specification, the heterocyclyl groups may be a monocyclic, bicyclic, tricyclic or tetracyclic ring system, which may include fused or bridged ring systems; and the nitrogen, carbon or sulfur atoms in the heterocyclyl group may be optionally oxidized; the nitrogen atom may be optionally quaternized; and the heterocyclyl group may be partially or fully saturated. Non-limiting examples include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

The term “pharmaceutically acceptable salts” includes both acid and base addition salts. The acid addition salt form of a compound that occurs in its free form as a base can be obtained by treating said free base form with an appropriate acid such as an inorganic acid or an organic acid. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Compounds containing acidic protons may be converted into their base addition salt form by treatment with appropriate organic and inorganic bases. Non-limiting examples of pharmaceutically acceptable base addition salts include sodium, potassium, lithium, ammonium (substituted and unsubstituted), calcium, magnesium, iron, zinc, copper, manganese, aluminum salts, N-methyl-D-glucamine salts, hydrabamine salts, and salts with amino acids such as, arginine, lysine and the like. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g. diastereomers and enantiomers, of the compounds. Furthermore, the present disclosure includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g. fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

### Methods for Characterizing Selectin Modulators

Biological activity of the selectin modulators described herein may be determined, for example, by performing at least one in vitro and/or in vivo study routinely practiced in the art and described herein or in the art.

In vitro assays include without limitation binding assays, immunoassays, competitive binding assays, and cell based activity assays. Selectin modulators as described above are capable, for example, of inhibiting selectin-mediated cell adhesion. This ability may generally be evaluated using any of a variety of in vitro assays designed to measure the effect on adhesion between selectin-expressing cells (e.g., adhesion between leukocytes or tumor cells and platelets or endothelial cells). For example, such cells may be plated under standard conditions that, in the absence of modulator, permit cell adhesion. In general, a selectin modulator is an inhibitor of selectin-mediated cell adhesion if contact of the test cells with the selectin modulator results in a discernible inhibition of cell adhesion. For example, in the presence of selectin modulators (e.g., micromolar levels), disruption of adhesion between leukocytes or tumor cells and platelets or endothelial cells may be determined visually within approximately several minutes, by observing the reduction of cells interacting with one another.

Selectin modulators may also be used in vitro, e.g., within a variety of well-known cell culture and cell separation methods. For example, modulators may be linked to the interior surface of a tissue culture plate or other cell culture support, for use in immobilizing selectin-expressing cells for screens, assays and growth in culture. Such linkage may be performed by any suitable technique. Selectin modulators may also be used, for example, to facilitate cell identification and sorting in vitro, permitting the selection of cells expressing a selectin (or different selectin levels). In some embodiments, the modulator(s) for use in such methods are linked to a detectable marker. Suitable markers are well known in the art and include radionuclides, luminescent groups, fluorescent groups, enzymes, dyes, constant immunoglobulin domains and biotin. In some embodiments, a selectin modulator linked to a fluorescent marker, such as fluorescein, is contacted with the cells, which are then analyzed by fluorescence activated cell sorting (FACS).

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing selectin modulators include animal model studies. Non-limiting examples of animal models for liquid cancers (such as lymphomas, leukemias, and myelomas) used in the art include multiple myeloma (see, e.g., DeWeerdt, Nature 480:S38-S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 Apr. 1; 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

### Exemplary Uses

In certain aspects, the compounds and compositions as described herein can be used to treat patients suffering from a condition associated with a seletin-mediated function. A variety of conditions are associated with a selectin-mediated function. Such conditions include, for example, tissue transplant rejection including graft versus host disease (GVHD), platelet-mediated diseases (e.g., atherosclerosis and clotting), sickle cell disease, vaso-occlusive disorders, sinusoidal obstruction syndrome, epilepsy, hyperactive coronary circulation, acute leukocyte-mediated lung injury (e.g., adult respiratory distress syndrome (ARDS)), Crohn's disease, inflammatory diseases (e.g., inflammatory bowel disease), autoimmune diseases (MS, myasthenia gravis), infection, cancer including blood cancers such as AML, ALL, CML, and MM (and metastasis), thrombosis, wounds (and wound-associated sepsis), burns, spinal cord damage, digestive tract mucous membrane disorders (gastritis, ulcers), osteoporosis, rheumatoid arthritis, osteoarthritis, asthma, allergy, psoriasis, septic shock, traumatic shock, stroke, nephritis, atopic dermatitis, frostbite injury, adult dyspnoea syndrome, ulcerative colitis, systemic lupus erythematosus, diabetes and reperfusion injury following ischaemic episodes. Selectin modulators may also be administered to a patient prior to heart surgery to enhance recovery. Other uses include pain management, prevention of restinosis associated with vascular stenting, and for undesirable angiogenesis, e.g., associated with cancer.

In certain aspects, the compounds and compositions as described herein can be used to treat patients suffering from sickle cell disease (SCD) or complications associated therewith. Sickle cell disease is an inheritable hematological disorder based on a mutation in the β-globin gene of hemoglobin. Upon deoxygenation, this mutated hemoglobin polymerizes and causes a shape change (sickling) of the red blood cell. This change in red blood cells leads to obstruction of blood vessels (vaso-occlusion) causing a wide variety of complications such as stroke, pulmonary hypertension, end-organ disease and death. Vaso-occlusive phenomena and hemolysis are clinical hallmarks of SCD. Vaso-occlusion results in recurrent painful episodes (sometimes called sickle cell crisis or vaso-occlusive crisis) and a variety of serious organ system complications among which, infection, acute chest syndrome, stroke, splenic sequestration are among the most debilitating. Vaso-occlusion accounts for 90% of hospitalizations in children with SCD, and it can ultimately lead to life-long disabilities and/or early death.

In addition to the fatal or potentially fatal complications, there are serious non-fatal complications of sickle cell disease such as pain. The severity of the pain may vary, but normally requires some form of medical attention. Hospitalization may be necessary.

In the U.S. alone, approximately 70,000-80,000 people suffer from sickle cell disease. Sickle cell disease is estimated to affect one of every 1,300 infants in the general population, and one of every 400 of African descent. Currently, there is no cure for sickle cell disease. The disease is chronic and lifelong. Life expectancy is typically shortened.

In certain aspects, the compounds and compositions as described herein can be used to treat patients suffering from graft versus host disease or complications associated therewith Graft-versus-host disease, GVHD, is an immunological disorder that is the major factor that limits the success and availability of allogeneic bone marrow or stem cell transplantation (collective referred to herein as allo-BMT) for treating some forms of otherwise incurable hematological malignancies, such as leukemia. GVHD is a systemic inflammatory reaction which causes chronic illness and may lead to death of the host mammal. At present, allogeneic transplants invariably run a severe risk of associated GVHD, even where the donor has a high degree of histocompatibility with the host. GVHD is caused by donor T-cells reacting against systemically distributed incompatible host antigens, causing powerful inflammation. In GVHD, mature donor T-cells that recognize differences between donor and host become systemically activated.

In certain aspects, the compounds and compositions as described herein can be used to treat patients suffering from sinusoidal obstruction syndrome (SOS) or complications associated therewith. SOS, also known as hepatic venoocclusive disease, was first diagnosed in cases of liver disease caused by the ingestion of herbal teas or food sources containing pyrrolizidine alkaloids from Crotalaria, Heliotropium and Senecio or from the consumption of bread made from inadequately winnowed wheat contaminated by seeds from these plants. With the modern development of chemotherapy, cases of SOS developed from the long-term use of azathioprine for immunosuppression after renal and liver transplantation and from the use of chemotherapeutic agents. Liver complications of chemotherapy are seen most commonly after high dose chemotherapy, with or without total body irradiation, or high dose radiation to the liver. Liver toxicity is not an uncommon side effect of high-dose chemotherapy. Liver toxicity also occurs after chemotherapy and/or liver irradiation when there is no bone marrow transplantation and hence, conditioning regimens used for marrow ablation are the most common cause of SOS. SOS is a common complication of chemotherapy with gemtuzumab ozogamicin or actinomycin D, or after long-term immunosuppression with azathioprine in kidney or liver transplantation patients. Other chemotherapeutic agents associated with liver toxicity and SOS include dacarbazine, cytosine arabinoside, mithrarnycin, 6-thioguanine, urethane, indicine N-oxide, alone and in combination. Milder forms of liver disease from chemotherapy which share the key aspect of sinusoidal endothelial cell injury include nodular regenerative hyperplasia, sinusoidal dilatation and peliosis hepatis. Combinations of irradiation and chemotherapy have also led to the development of SOS. For example, treating nephroblastoma (Wilms' tumor) with dactinomycin and abdominal irradiation has led to SOS.

Radiation-induced liver disease is a condition that shares some of the features of SOS, although there are differences in clinical presentation, histology and time course. Radiation-induced liver disease is seen with radiation doses in excess of 30 to 35 Gy in adults.

SOS has significant morbidity and mortality. The severity of SOS can be classified as mild (SOS is clinically obvious, but requires no treatment and resolves completely), moderate (SOS that causes signs and symptoms requiring treatment such as diuretics or pain medications, but resolves completely) or severe (SOS that requires treatment but that does not resolve before death or day 100. Some patients have subclinical liver damage, evinced by histologic evidence of liver toxicity in the absence of clinical signs and symptoms. Despite deep jaundice, patients with severe SOS seldom die of liver failure, but rather from renal and cardiopulmonary failure.

A clinically useful model for predicting the outcome of SOS after cyclophosphamide-based regimes is derived from rates of increase of both bilirubin and weight in the first two weeks following transplantation. Furthermore, a poor prognosis correlates with higher serum AST and ALT values, higher wedged hepatic venous pressure gradient, development of portal vein thrombosis, doubling of the baseline serum creatinine, and decreasing oxygen saturation. There is currently no prophylactic treatment for either SOS or radiation-induced liver disease, and there are no proven therapeutic remedies with high efficacy. The only therapeutic modality with some proven efficacy is the combination of heparin plus tissue plasminogen activator. However, this combination can only be safely used in a very limited group of patients and has efficacy in less than 30% of this limited population of patients.

SOS is the dose-limiting toxicity for several chemotherapeutic drugs and limits patient eligibility. A prophylactic treatment of SOS would have a significant impact on the ability to use high dose chemotherapy. Development of therapies to treat SOS after onset of the disease would be of value in unexpected cases of chemotherapy-induced liver disease.

The molecular events have been best characterized in the rat monocrotaline model. Monocrotaline, the pyrrolizidine alkaloid found in Crotalaria, is one of the best-studied toxins involving SOS. The monocrotaline model of SOS has the same histologic characteristics as the human disease, as well as the same “clinical features,” i.e., hyperbilirubinemia, hepatomegaly, and ascites formation. In this model, the first morphologic change noted by electron microscopy is loss of thesinusoidal endothelial cell fenestration and the appearance of gaps in the sinusoidal endothelial cell barrier. Studies with in vivo microscopy and confirmation by electron microscopy have shown that sinusoidal endothelial cells round up, and red blood cells begin to penetrate into the space of Disse beneath the rounded up endothelial cells and dissect off the sinusoidallining. The sloughed sinusoidal lining cells (i.e., Kupffer cells, sinusoidal endothelial cells, and stellate cells) embolize downstream and obstruct sinusoidal flow. By the time hepatocyte necrosis is observed, there is extensive denudation of the sinusoidal lining. Early on, there is loss of Kupffer cells, but there is a significant influx of monocytes within the sinusoids, which exacerbates the obstruction of sinusoidal flow by the embolized sinusoidal lining cells. The rounding up or swelling of sinusoidal endothelial cells is the initiating event in SOS and leads to dissection of the sinusoidal lining, which embolizes and blocks the microcirculation.

In certain aspects, the compounds and compositions as described herein can be used to treat patients suffering from cancers of the blood and complications associated therewith. Such cancer group includes hematological malignancies. Examples of cancers of the blood include acute myelogenous leukemia (AML), acute lymphoblastic leukemia (ALL), chronic myelogenous leukemia (CML) and multiple myeloma (MM).

Complications associated with a cancer of the blood include, for example, shortened life expectancy, organ damage, periodic or chronic pain, migration of cancer cells out of blood circulation, and reduction in red blood cells, white blood cells or platelets. It is desirable to prevent cancer cells from leaving the primary site, or to prevent extravasation of cancer cells from the bloodstream and infiltration into other tissues. Cancer cells while in the bloodstream are typically susceptible to chemotherapy, but are more difficult to treat once they leave the bloodstream. For example, cancer cells (such as MM cells) can extravasate from the bloodstream and infiltrate into bone marrow matrix where they are inaccessible to chemotherapeutic agents circulating in the bloodstream. Consequences of the complication of migration of cancer cells out of blood circulation include relapse (failure to cure) and disseminated disease (metastasis) leading, for example, to organ damage or failure. AML is an example of a blood cancer with the complication of migration of cancer cells out of blood circulation resulting in disseminated disease.

Acute myelogenous leukemia (also known as acute myeloid leukemia or AML) is a cancer of white blood cells, and in particular the myeloid line. It appears that AML arises from a single progenitor cell which has undergone genetic transformation to an abnormal cell with the ability to proliferate rapidly. These abnormal immature myeloid cells accumulate in the bone marrow. This accumulation in the bone marrow interferes with the production of normal blood cells, including a reduction in red blood cells, platelets and neutrophils. Eventually the bone marrow stops working correctly.

AML is one of the most common types of leukemia among adults, and the most common acute leukemia affecting adults. In the U.S. alone, there are approximately 12,000 new cases each year. The incidence of AML is expected to increase as the population ages. In addition, in the U.S., about 11% of the cases of leukemia in childhood are AML. Chemotherapy is generally used to treat AML. Only a minority of patients are cured with current therapy.

Chemotherapy has a number of deleterious side effects. One of the side effects is myeloablative bone marrow toxicities. Bone marrow is the tissue that fills the inside of some bones. Examples of such bones are sternum, hip, femur and humerus. Bone marrow contains stem cells that develop into several types of blood cells: erythrocytes (red blood cells), leukocytes (white blood cells) and thrombocytes (platelets). Cells in the bone marrow are susceptible to the effects of chemotherapy due to their rapid rate of division. Bone marrow is prevented by chemotherapeutic agents from forming new blood cells. With time after exposure to a chemotherapeutic agent, counts of the blood cells will fall at various rates, depending upon the particular type of cell as their average life spans differ. Low white blood cell count, for example, makes an individual more susceptible to infection. Low red blood cell count, for example, causes an individual to be fatigued. Low platelet count, for example, impairs an individual's ability to make a blood clot.

In certain aspects, the compounds and compositions as described herein can be used to treat patients suffering from epilepsy. Epilepsy is one of the most common neurological problems, with up to about 1% of the population afflicted. Epileptogenesis is a term used to refer to the process of a normal brain becoming epileptic in the first place. In the process (which may occur after acute brain injury), lesions and changes in the brain progress to the formation of chronic seizures. Acute injury to the brain can arise, for example, from traumatic physical brain injury (i.e., closed head injury), stroke or infection. The term epileptogenesis is also used for the process of how a mildly epileptic brain can worsen. While the reduction or prevention of seizures has understandably been the focus of substantial medical research, one ultimately would like to prevent epilepsy or stop its progression by the development of an anti-epileptogenic agent.

The term “epilepsy” as commonly used includes more than one type of disorder, and in its generic meaning is better termed “epileptic syndromes.” An example of an epileptic syndrome is Rasmussen's syndrome.

Rasmussen's syndrome was first described in 1958 and remains an unresolved medical problem. This devastating disorder afflicts mainly children and can destroy a cerebral hemisphere. Progressive neurological deterioration (including brain atrophy) and seizures are associated with Rasmussen's syndrome. Medical treatment has typically included anticonvulsant therapy and hemispherectomy surgery where half of the brain is removed. The surgery has been more effective than anti-seizure drugs in stopping the seizures. However, side effects of the surgery typically include the addition of a limp to walking and running, and on the side opposite to the surgery there is significant impairment of hand function and loss of fine motor skills.

In some embodiments, a compound of Formula (I) and/or a pharmaceutical composition comprising at least one compound of Formula (I) may be used for treating at least one of the diseases, disorders, and conditions described herein or for the preparation or manufacture of a medicament for use in treating at least one of the diseases, disorders, and/or conditions described herein. Each of these methods and uses is described in greater detail.

As understood by a person of ordinary skill in the medical art, the terms, “treat” and “treatment,” include medical management of a disease, disorder, or condition of a subject (i.e., patient, individual) (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment. Subjects in need of treatment include those who already have the disease, condition, or disorder as well as subjects prone to have or at risk of developing the disease, condition, or disorder, and those in which the disease, condition, or disorder is to be prevented (i.e., decreasing the likelihood of occurrence of the disease, disorder, or condition).

In some embodiments of the methods described herein, the subject is a human. In some embodiments of the methods described herein, the subject is a non-human animal. A subject in need of treatment as described herein may exhibit at least one symptom or sequelae of the disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing a disease, disorder, or condition described herein can readily be determined by a person of ordinary skill in the medical and clinical arts. Determining and adjusting an appropriate dosing regimen (e.g., adjusting the amount of compound per dose and/or number of doses and frequency of dosing) can also readily be performed by a person of ordinary skill in the medical and clinical arts. One or any combination of diagnostic methods, including physical examination, assessment and monitoring of clinical symptoms, and performance of analytical tests and methods described herein, may be used for monitoring the health status of the subject.

### Pharmaceutical Compositions and Methods of Using Pharmaceutical Compositions

Also provided herein are pharmaceutical compositions comprising at least one compound of Formula (I). In some embodiments, the pharmaceutical composition further comprises at least one additional pharmaceutically acceptable ingredient.

In pharmaceutical dosage forms, any one or more of the compounds of the present disclosure may be administered in the form of a pharmaceutically acceptable derivative, such as a salt, and/or it/they may also be used alone and/or in appropriate association, as well as in combination, with other pharmaceutically active compounds.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject.

In general, the amount of at least one compound of Formula (I) as described herein, that is present in a dose, may range from about 0.01 μg to about 1000 μg per kg weight of the subject. The minimum dose that is sufficient to provide effective therapy may be used in some embodiments. Subjects may generally be monitored for therapeutic effectiveness using assays suitable for the disease or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease or disorder may be determined according to parameters understood by a person of ordinary skill in the medical art.

Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical composition described herein may be sterile aqueous or sterile non-aqueous solutions, suspensions or emulsions, and may additionally comprise at least one pharmaceutically acceptable excipient (i.e., a non-toxic material that does not interfere with the activity of the active ingredient). Such compositions may be in the form of a solid, liquid, or gas (aerosol). Alternatively, the compositions described herein may be formulated as a lyophilizate, or compounds described herein may be encapsulated within liposomes using technology known in the art. The pharmaceutical compositions may further comprise at least one additional pharmaceutical acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient is selected based on the mode of administration, as well as the chemical composition of the active ingredient(s). Pharmaceutical compositions may be formulated for the particular mode of administration. For parenteral administration, pharmaceutical compositions may further comprise water, saline, alcohols, fats, waxes, and buffers. For oral administration, pharmaceutical compositions may further comprise at least one ingredient chosen, for example, from any of the aforementioned excipients, solid excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid pharmaceutical composition may include, for example, at least one the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition an injectable pharmaceutical composition, and in some embodiments, the injectable pharmaceutical composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound or composition comprising the same.

### General Synthetic Methodology

The compounds of this invention may be prepared in general by methods known to those skilled in the art. Schemes 1-5 below provide general synthetic routes for the preparation of compounds disclosed herein. Other equivalent schemes, which will be readily apparent to the ordinary skilled organic chemist, may alternatively be used to synthesize various portions of the molecules as illustrated by the general schemes below.

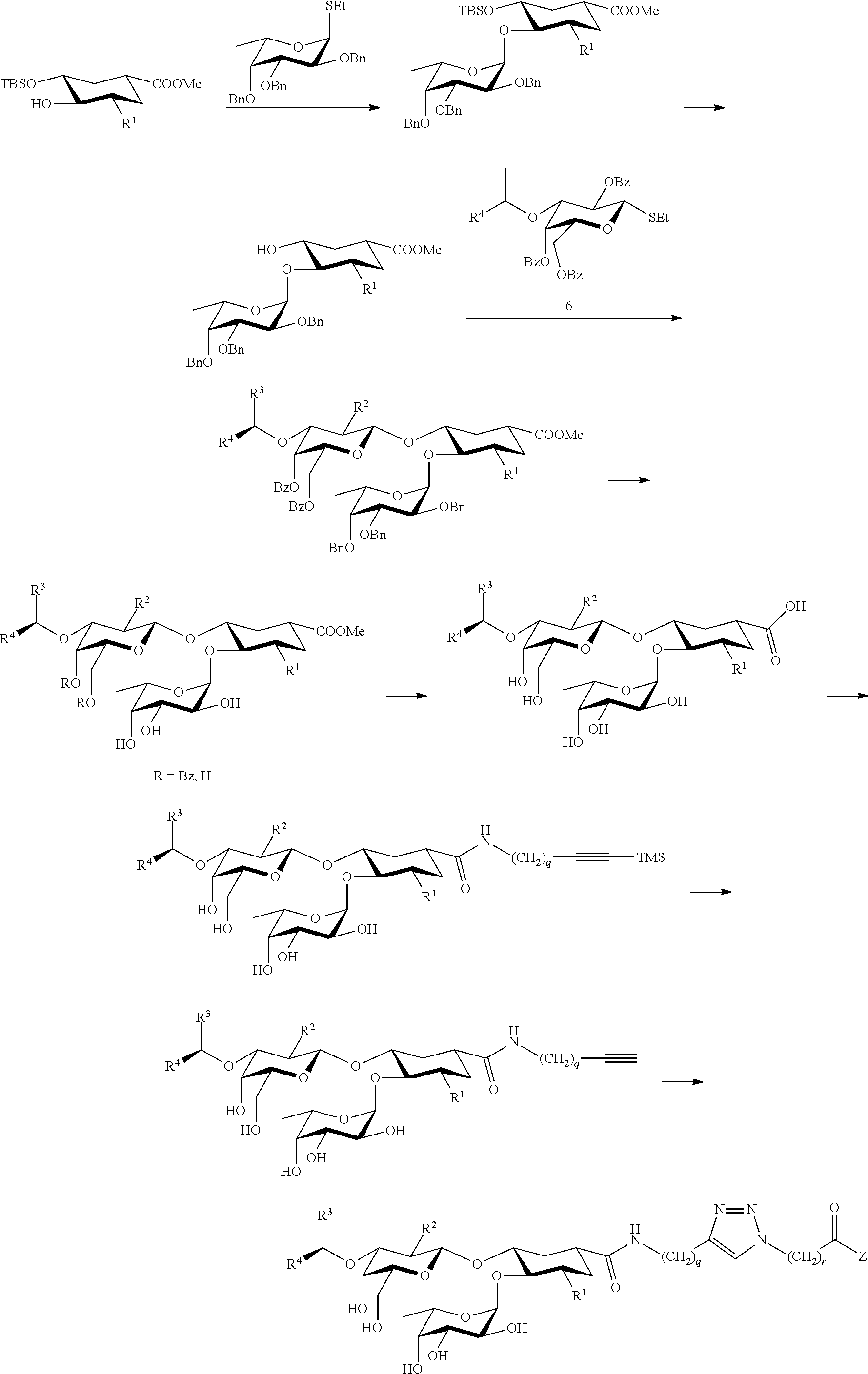

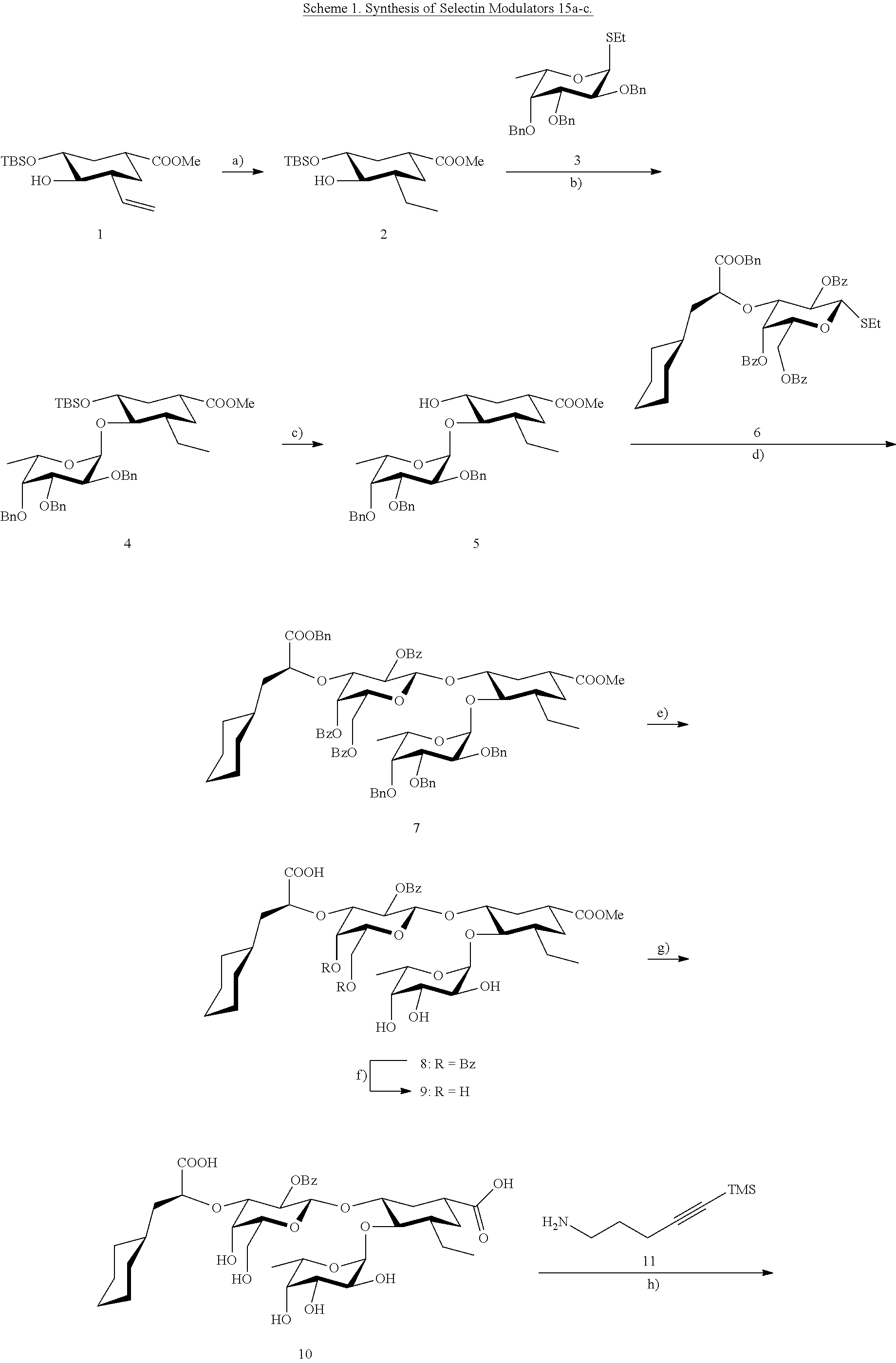

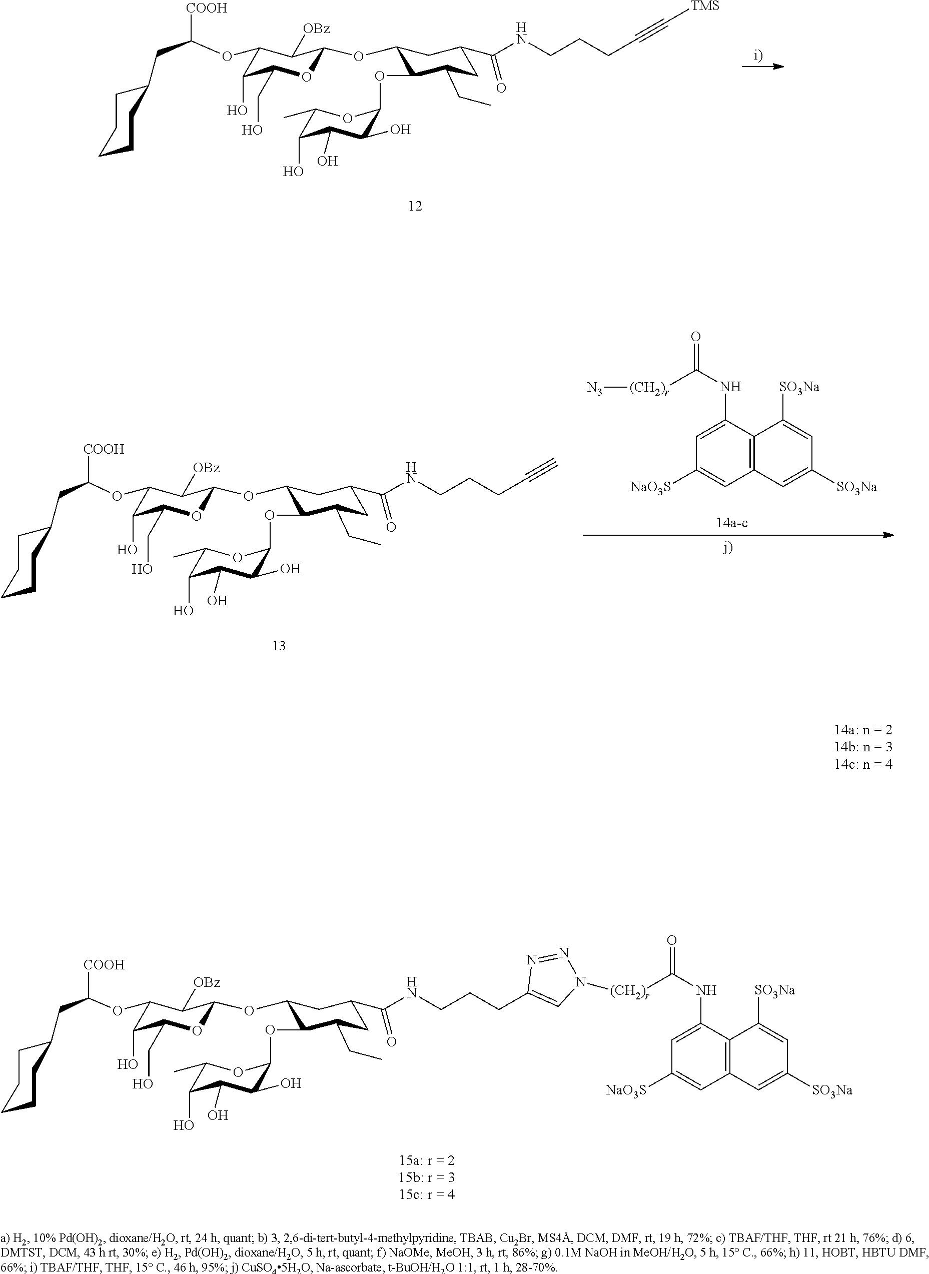

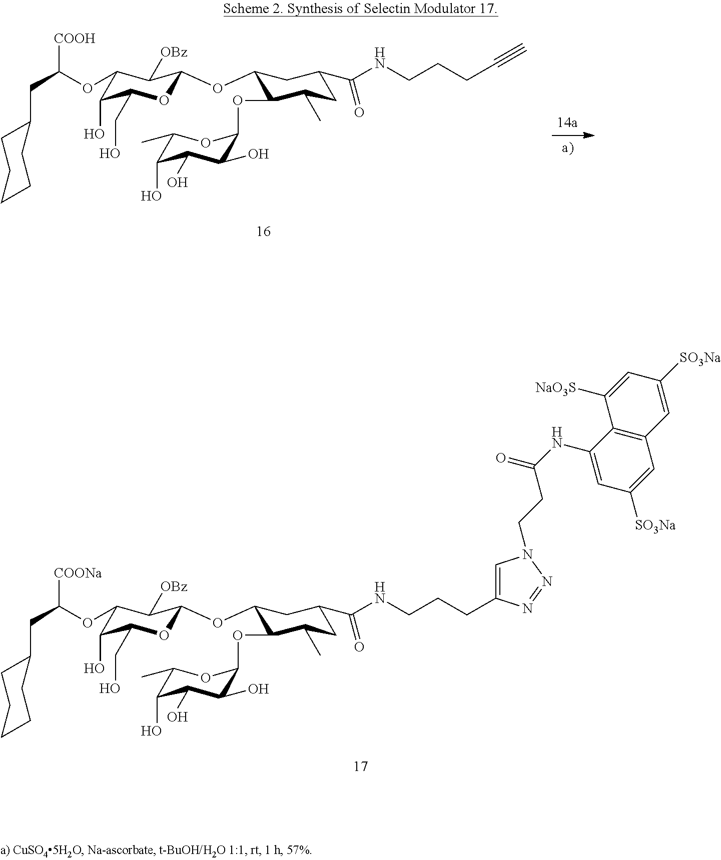

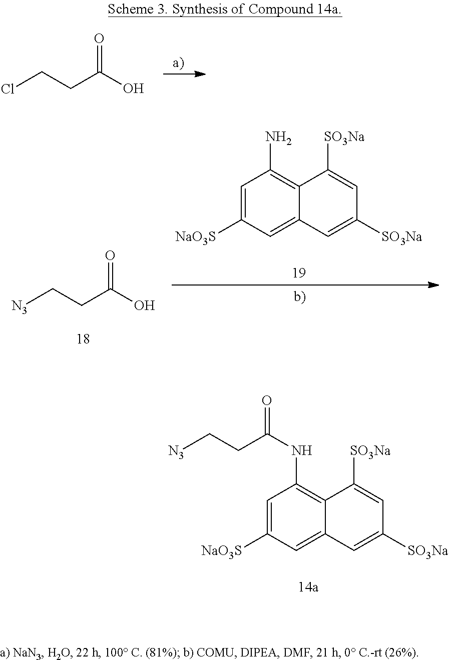

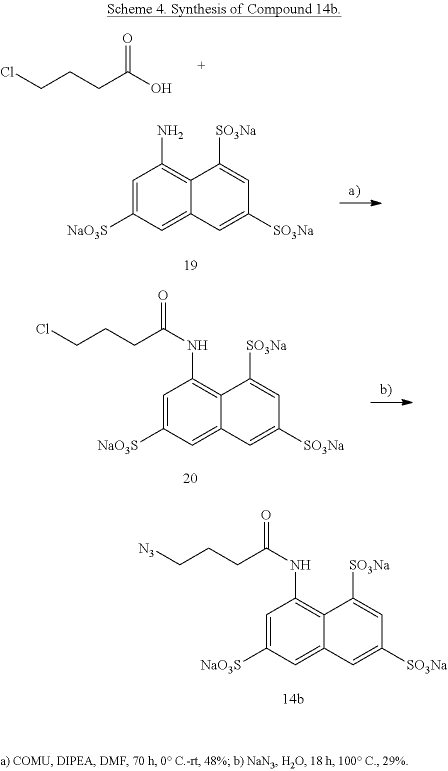

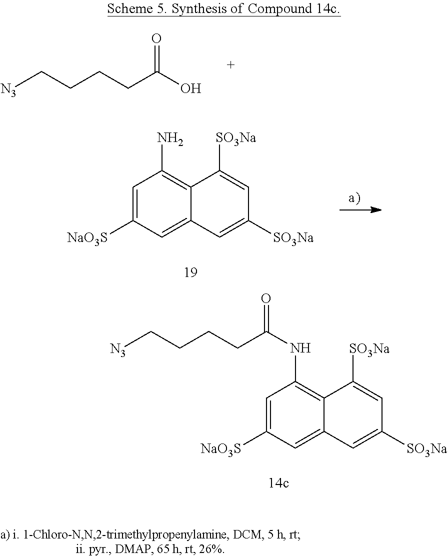

### EXAMPLES

### Example 1

### Synthesis of Seletin Modulators 15a-c (FIG. 1)

Synthesis of Compound 2:

A suspension of compound 1 (500 mg, 1.19 mmol, purity approximately 75%; synthesized as described by Schwizer et al., Chem. Eur. J. 18:1342-1351, 2012) and Pd(OH)₂/C (10%) in dioxane/water (4:1, 5 mL) is stirred under a hydrogen atmosphere at room temperature for 24 hours. Then the reaction mixture is filtered over celite and washed with methanol. The filtrate is evaporated to dryness and the crude product is purified by column chromatography on silica gel (petroleum ether/diethyl ether, 9:1) to afford compound 2 (360 mg, quant.) as a yellowish oil. [α]_{D}²⁰ −16.5 (c 1.14, CH₂Cl₂); ¹H NMR (500 MHz, CDCl₃): δ=0.10 (s, 3H, SiCH₃), 0.10 (s, 3H, SiCH₃), 0.90 (s, 9H, C(CH₃)₃), 0.89-0.93 (m, 3H, CH₃), 1.12-1.22 (m, 2H, H-6a, OH), 1.26 (m, 1H, CH₂), 1.37 (m, 1H, H-5), 1.53 (m, 1H, H-2a), 1.85 (m, 1H, CH₂), 2.01 (m, 1H, H-6b), 2.07 (ddd, J=4.0, 6.2, 12.7 Hz, 1H, H-2b), 2.40 (m, 1H, H-1), 3.07 (dd, J=8.6, 10.1 Hz, 1H, H-4), 3.42 (ddd, J=4.5, 8.4, 11.4 Hz, 1H, H-3), 3.68 (s, 3H, OCH₃); ¹³C NMR (126 MHz, CDCl₃): δ=−4.74, −4.10 (2 SiCH₃), 10.62 (CH₃), 17.96 (C(CH₃)₃), 24.31 (CH₂), 25.77 (C(CH₃)₃), 31.81 (C-6), 35.60 (C-2), 40.66 (C-1), 41.78 (C-5), 51.79 (OCH₃), 75.82 (C-3), 78.17 (C-4), 175.00 (C═O); ESI-MS: m/z: Calcd for C₁₆H₃₃O₄Si [M+H]⁺: 317.21, found: 317.09.

Synthesis of Compound 4:

Compound 3 (1.36 g, 2.84 mmol) and (Bu)₄NBr (1.37 g, 4.27 mmol) are dried at high vacuum overnight. Then, compound 2 (450 mg, 1.42 mmol), 2,6-di-tert-butyl-4-methylpyridine (875 mg, 4.27 mmol), powdered 4 Å molecular sieves (1.5 g), anhydrous DCM (10 mL) and DMF (1.5 mL) are added and the mixture is stirred at room temperature under argon for 5 hours. CuBr₂ (953 mg, 4.27 mmol), dried at 70° C. under high vacuum overnight, is added and stirring is continued for another 20 hours. The reaction mixture is then filtered through a pad of celite, the filtrate is diluted in DCM and successively washed with a solution of saturated aqueous NH₄Cl and aqueous NH₃ (9:1, v/v) (2×100 mL) and brine (100 mL). The aqueous layers are then extracted with DCM (2×100 mL), and the combined organic layers are dried (Na₂SO₄) and concentrated in vacuo. The crude product is purified by column chromatography on silica gel (petroleum ether/EtOAc, 92.5:7.5) to afford compound 4 (780 mg, 78%) as a yellowish oil. [α]_{D}²⁰ −72.8 (c 0.70, CH₂Cl₂); ¹H NMR (500 MHz, CDCl₃): δ=0.02 (s, 3H, SiCH₃), 0.07 (s, 3H, SiCH₃), 0.83 (t, J=7.4 Hz, 3H, CH₃), 0.87 (s, 9H, C(CH₃)₃), 1.09 (d, J=6.5 Hz, 3H, H-6^{F}), 1.13-1.31 (m, 2H, H-6a, CH₂), 1.49 (m, 1H, H-5), 1.58 (m, 1H, H-2a), 1.83-1.98 (m, 2H, H-6b, CH₂), 2.05 (m, 1H, H-2b), 2.36 (tt, J=3.8, 12.2 Hz, 1H, H-1), 3.37 (m, 1H, H-4), 3.64 (m, 1H, H-4^{F}), 3.67 (s, 3H, OCH₃), 3.68 (m, 1H, H-3), 4.01-4.08 (m, 2H, H-2^{F}, H-3^{F}), 4.35 (q, J=6.1 Hz, 1H, H-5^{F}), 4.64 (A of AB, J=11.6 Hz, 1H, CH₂Ph), 4.72 (A of AB, J=11.8 Hz, 1H, CH₂Ph), 4.73 (A of AB, J=11.8 Hz, 1H, CH₂Ph), 4.82 (B of AB, J=11.9 Hz, 1H, CH₂Ph), 4.85 (B of AB, J=11.9 Hz, 1H, CH₂Ph), 4.98 (B of AB, J=11.5 Hz, 1H, CH₂Ph), 5.14 (d, J=2.8 Hz, 1H, H-1^{F}), 7.22-7.43 (m, 15H, 3 C₆H₅); ¹³C NMR (126 MHz, CDCl₃): δ=−4.97, −3.36 (2 SiCH₃), 10.79 (CH₃), 16.96 (C-6^{F}), 18.15 (C(CH₃)₃), 24.16 (CH₂), 26.15 (C(CH₃)₃), 31.52 (C-6), 37.05 (C-2), 40.40 (C-1), 43.54 (C-5), 51.72 (OCH₃), 66.36 (C-5^{F}), 72.81 (CH₂Ph), 74.19 (C-3), 74.42, 74.88 (CH₂Ph), 76.62 (C-2^{F}), 78.28 (C-4^{F}), 79.27 (C-3^{F}), 80.44 (C-4), 97.24 (C-1¹′), 127.42, 127.46, 127.60, 128.14, 128.22, 128.26, 128.33, 138.57, 138.86, 138.94 (18C, 3 C₆H₅), 175.13 (C═O); ESI-MS: m/z: Calcd for C₄₃H₆₀NaO₈Si [M+Na]⁺: 755.40, found: 755.43.

Synthesis of Compound 5:

TBAF (1 M in THF, 5.0 mL, 5 mmol) is added to a solution of compound 4 (780 mg, 1.06 mmol) in anhydrous THF (6.4 mL). After stirring at room temperature for 20 hours, the reaction mixture is diluted with DCM (100 mL) and washed with water (2×100 mL) and brine (100 mL). The aqueous layers are extracted with DCM (2×50 mL) and the combined organic layers are dried (Na₂SO₄) and concentrated. The crude product is purified by column chromatography on silica gel (petroleum ether/EtOAc, 4:1) to afford compound 5 (500 mg, 76%) as a white solid. [α]_{D}²⁰ −74.6 (c 1.16, CH₂Cl₂); ¹H NMR (500 MHz, CDCl₃): δ=0.72 (t, J=7.5 Hz, 3H, CH₃), 1.00-1.12 (m, 2H, H-6a, CH₂), 1.09 (d, J=6.5 Hz, 3H, H-6^{F}), 1.28-1.44 (m, 2H, H-2a, H-5), 1.92-2.02 (m, 2H, H-6b, CH₂), 2.16 (m, 1H, H-2b), 2.28 (tt, J=3.1, 12.6 Hz, 1H, H-1), 2.91 (dd, J=8.7, 10.1 Hz, 1H, H-4), 3.36 (ddd, J=4.8, 8.4, 11.6 Hz, 1H, H-3), 3.60 (s, 3H, OCH₃), 3.63 (d, J=1.4 Hz, 1H, H-4^{F}), 3.89 (dd, J=2.7, 10.2 Hz, 1H, H-3^{F}), 4.00-4.07 (m, 2H, H-2^{F}, H-5^{F}), 4.58 (A of AB, J=11.5 Hz, 1H, CH₂Ph), 4.62 (A of AB, J=11.7 Hz, 1H, CH₂Ph), 4.67-4.79 (m, 3H, CH₂Ph), 4.89-4.94 (m, 2H, H-1^{F}, CH₂Ph), 7.16-7.39 (m, 15H, 3 C₆H₅); ¹³C NMR (126 MHz, CDCl₃): δ=10.80 (CH₃), 16.60 (C-6^{F}), 23.92 (CH₂), 32.08 (C-6), 34.68 (C-2), 40.33 (C-1), 41.52 (C-5), 51.74 (OCH₃), 67.69 (C-5^{F}), 72.15 (C-3), 72.85, 73.63, 74.88 (3 CH₂Ph), 76.27 (C-2^{F}), 77.42 (C-4^{F}), 78.83 (C-3^{F}), 90.10 (C-4), 98.64 (C-1^{F}), 127.41, 127.50, 127.57, 127.66, 127.83, 128.23, 128.25, 128.35, 138.27, 138.40, 138.67 (18C, 3 C₆H₅), 175.05 (C═O); ESI-MS: m/z: Calcd for C₃₇H₄₆NaO₈ [M+Na]⁺: 641.31, found: 641.23.

Synthesis of Compound 7:

A mixture of compound 5 (480 mg, 0.775 mmol), compound 6 (908 mg, 1.16 mmol), and activated powdered molecular sieves 4 Å (400 mg) in DCM (5 mL) is stirred at room temperature under argon for 4 hours. A pre-stirred mixture (4 hours at room temperature) of DMTST (800 mg, 3.10 mmol) and activated powdered MS 4 Å (200 mg) in DCM (3 mL) is added. After 43 hours, the reaction mixture is filtered over celite, diluted with DCM (100 mL), and washed with saturated aqueous NaHCO₃ (100 mL) and brine (100 mL). The aqueous layers are extracted with DCM (2×100 mL) and the combined organic layers are dried (Na₂SO₄) and evaporated to dryness. The crude product is purified by column chromatography on silica gel (toluene/EtOAc, 9:1) to afford compound 7 as a white solid (302 mg, 30%). [α]_{D}²⁰ −66.2 (c 0.63, CH₂Cl₂); ¹H NMR (500 MHz, CDCl₃): δ=0.32-0.50, 0.55-1.34 (m, 17H, C₇H₁₃^{L}, CH₂, H-2a, H-5, H-6a), 0.68 (t, J=7.4 Hz, 3H, CH₃) 1.36 (d, J=6.5 Hz, 3H, H-6^{F}), 1.79 (dd, J=2.5, 13.3 Hz, 1H, H-6b), 1.89 (m, 1H, CH₂), 2.05-2.14 (m, 2H, H-1, H-2b), 3.21 (t, J=9.6 Hz, 1H, H-4), 3.42-3.50 (m, 2H, H-3, H-4^{F}), 3.46 (s, 3H, OCH₃), 3.75 (dd, J=3.4, 9.9 Hz, 1H, H-3^{G}), 3.85 (t, J=6.5 Hz, 1H, H-5^{G}), 3.93-3.98 (m, 2H, H-2^{F}, H-3^{F}), 4.05 (dd, J=4.6, 7.9 Hz, 1H, H-2^{L}), 4.17-4.22 (m, 2H, H-6a^{G}, CH₂Ph), 4.31 (dd, J=5.6, 11.5 Hz, 1H, H-6b^{G}), 4.45 (A of AB, J=11.8 Hz, 1H, CH₂Ph), 4.49 (d, J=8.2 Hz, 1H, H-1^{G}), 4.55 (B of AB, J=11.7 Hz, 1H, CH₂Ph), 4.57 (A of AB, J=11.5 Hz, 1H, CH₂Ph), 4.67 (B of AB, J=11.4 Hz, 1H, CH₂Ph), 4.73 (B of AB, J=11.4 Hz, 1H, CH₂Ph), 4.82 (q, J=6.4 Hz, 1H, H-5^{F}), 4.91 (m, 1H, H-1^{F}), 4.97 (A of AB, J=12.1 Hz, 1H, CH₂Ph), 5.05 (B of AB, J=12.1 Hz, 1H, CH₂Ph), 5.54 (dd, J=8.6, 9.5 Hz, 1H, H-2^{G}), 5.77 (d, J=3.0 Hz, 1H, H-4^{G}), 7.07-7.28, 7.29-7.42, 7.43-7.53, 7.95-8.07 (m, 35H, 7 C₆H₅); ¹³C NMR (126 MHz, CDCl₃): δ=10.47 (CH₃), 16.78 (C-6^{F}), 23.05, 23.80, 25.46, 25.70, 26.06, 30.87, 32.62, 33.20, 33.38, 40.23, 40.44, 44.05 (C₇H₁₃^{L}, CH₂, C-1, C-2, C-5, C-6), 51.63 (OCH₃), 62.50 (C-6^{G}), 66.43 (C-5^{F}), 66.63 (CH₂Ph), 70.20 (C-4^{G}), 71.61 (C-5^{G}), 72.00 (2C, C-2^{G}, CH₂Ph), 74.55, 74.89 (2 CH₂Ph), 76.32 (C-2^{F}), 78.07 (C-3^{G}), 78.44 (C-2^{L}), 78.67 (C-4), 79.23 (C-4^{F}), 79.90 (C-3^{F}), 80.42 (C-3), 98.23 (C-1^{F}), 100.17 (C-1^{G}), 126.92, 127.05, 127.18, 127.43, 127.69, 128.02, 128.08, 128.11, 128.40, 128.43, 128.50, 128.54, 129.61, 129.67, 129.75, 129.91, 129.94, 133.04, 133.18, 133.27, 135.41, 138.58, 138.95, 139.18 (42C, 7 C₆H₅), 164.60, 166.09, 166.14, 172.39, 174.25 (5 C═O); ESI-MS: m/z: Calcd for C₈₀H₈₈NaO₁₈ [M+Na]⁺: 1359.59, found: 1359.84.

Synthesis of Compound 8:

A mixture of 7 (204 mg, 0.153 mmol) and Pd(OH)₂/C (45 mg) in dioxane/H₂O (4:1, 3 mL) is stirred at room temperature under a hydrogen atmosphere. After 5 hours, the reaction mixture is filtered through a pad of celite and evaporated to dryness to afford compound 8 (150 mg, quant) as a white solid. ESI-MS: m/z: Calcd for C₅₂H₆₄NaO₁₈ [M+Na]⁺: 999.40, found: 999.49.

Synthesis of Compound 9:

To a mixture of 8 (150 mg, 0.153 mmol) in methanol (2 mL), a catalytic amount of freshly prepared sodium methoxide/methanol is added. After 3 hours, the reaction mixture is neutralized with acetic acid and evaporated to dryness. The residue is purified by column chromatography on silica gel (DCM/MeOH/H₂O, 10:3:0.5) to give compound 9 (102 mg, 86%) as a white solid. [α]_{D}²⁰ −80.5 (c 0.94, MeOH); ¹H NMR (500 MHz, CD₃OD): δ=0.42-1.15, 1.16-1.67 (m, 18H, C₇H₁₃^{L}, CH₂, H-2a, H-6a, H-5), 0.85 (t, J=7.4 Hz, 3H, CH₃), 1.33 (d, J=6.6 Hz, 3H, H-6^{F}), 1.87-2.01 (m, 2H, CH₂, H-6b), 2.27-2.41 (m, 2H, H-2b, H-1), 3.22 (dd, J=6.3, 13.0 Hz, 1H, H-4), 3.57 (s, 3H, OCH₃), 3.60 (m, 1H, H-5^{G}), 3.63 (dd, J=2.8, 9.8 Hz, 1H, H-3^{G}), 3.66-3.82 (m, 5H, H-3, H-2^{F}, H-4^{F}, H-6^{G}), 3.85-3.93 (m, 2H, H-2^{L}, H-3^{F}), 3.97 (d, J=0.8 Hz, 1H, H-4^{G}), 4.72 (d, J=8.1 Hz, 1H, H-1^{G}), 4.93 (d, J=4.0 Hz, 1H, H-1^{F}), 4.98 (m, 1H, H-5^{F}), 5.42 (dd, J=8.4, 9.4 Hz, 1H, H-2^{G}), 7.50 (t, J=7.8 Hz, 2H, C₆H₅), 7.60-7.66, 8.06-8.10 (m, 3H, C₆H₅); ¹³C NMR (126 MHz, CD₃OD): δ=10.75 (CH₃), 16.72 (C-6^{F}), 24.50 (CH₂), 26.55, 26.76, 27.33, 32.56, 33.08, 34.32, 34.88, 35.29, 41.30, 43.20, 45.01 (C₇H₁₃^{L}, C-1, C-2, C-5, C-6), 52.27 (OCH₃), 62.85 (C-6^{G}), 67.67 (C-4^{G}), 67.75 (C-5^{F}), 70.31 (C-2^{F}), 71.39 (C-3^{F}), 73.03 (C-2^{G}), 73.94 (C-4^{F}), 75.80 (C-5^{G}), 79.36 (C-2^{L}), 80.15 (C-3), 80.64 (C-4), 83.73 (C-3^{G}), 100.23 (C-1^{F}), 100.72 (C-1^{G}), 129.64, 130.92, 131.61, 134.30 (6C, C₆H₅), 166.92, 176.30 (3C, C═O); ESI-MS: m/z: Calcd for C₃₈H₅₆NaO₁₆ [M+Na]⁺: 791.35, found: 791.35.

Synthesis of Compound 10:

A solution of 1 M NaOH in H2O/MeOH (1:1, 725 μL) is cooled to 15° C. Then, compound 9 (25 mg, 0.0325 mmol) is added and the mixture is stirred at 15° C. After 4 hours, the reaction mixture is neutralized with 0.5 M aq. HCl and the solvents are removed in vacuo. The residue is purified by column chromatography on RP-18 (H₂O/MeOH, 0-10%) to afford compound 10 (16.3 mg, 66%) as a white solid. [α]_{D}²⁰ −76.4 (c 0.29, MeOH); ¹H NMR (500 MHz, CD₃OD): δ=0.41-0.54, 0.55-0.73, 0.79-0.93, 1.01-1.15, 1.15-1.43, 1.42-1.56, 1.64 (m, 20H, C₇H₁₃^{L}, CH₂, H-2a, H-5, H-6a, CH₃), 1.32 (d, J=6.5 Hz, 3H, H-6^{F}), 1.87 (dp, J=3.1, 13.5 Hz, 1H, H-6b), 1.94 (m, 1H, CH₂), 2.13 (tt, J=3.3, 12.8 Hz, 1H, H-1), 2.31 (ddd, J=3.1, 5.5, 12.0 Hz, 1H, H-2b), 3.27 (m, 1H, H-4), 3.57 (dd, J=3.1, 9.7 Hz, 1H, H-3^{G}), 3.60 (m, 1H, H-5^{G}), 3.67-3.77 (m, 4H, H-2^{L}, H-3, H-2^{F}, H-6a^{G}), 3.77 (d, J=3.3 Hz, 1H, H-4^{F}), 3.81 (dd, J=6.8, 11.6 Hz, 1H, H-6b^{G}), 3.88-3.96 (m, 2H, H-3^{F}, H-4^{G}), 4.75 (d, J=8.1 Hz, 1H, H-1^{G}), 4.90-4.99 (m, 2H, H-1^{F}, H-5^{F}), 5.42 (dd, J=9.7, 8.0 Hz, 1H, H-2^{G}), 7.43-7.53, 7.53-7.62, 7.97-8.16 (m, 5H, C₆H₅); ¹³C NMR (126 MHz, CD₃OD): δ=10.86 (CH₃), 16.76 (C-6^{F}), 24.87 (CH₂), 26.54, 26.78, 27.44, 33.07, 33.92, 34.40, 35.43, 36.08, 43.60, 45.34, 45.47 (C₇H₁₃^{L}, C-1, C-2, C-5, C-6), 63.23 (C-6^{G}), 67.74 (C-4^{G}), 67.84 (C-5^{F}), 70.39 (C-2^{F}), 71.32 (C-3^{F}), 72.98 (C-2^{G}), 73.99 (C-4^{F}), 75.96 (C-5^{G}), 80.32, 80.55 (C-3, C-2^{L}), 81.09 (C-4), 83.96 (C-3^{G}), 99.95 (C-1^{F}), 100.31 (C-1^{G}), 129.68, 130.94, 131.79, 134.11 (6C, C₆H₅), 167.06, 183.27, 183.45 (3 C═O); ESI-MS: m/z: Calcd for C₃₇H₅₄NaO₁₆ [M+Na]⁺: 777.33, found: 777.46.

Synthesis of Compound 12:

To a mixture of compound 10 (11 mg, 0.0146 mmol) in dry DMF (800 μL), HOBT (5.9 mg, 0.0437 mmol) and HBTU (6.6 mg, 0.0175 mmol) are added successively at 0° C. After stirring for 10 minutes, alkine 11 (47 μL) is added and the mixture is stirred for 1 hour at 0° C. The solvents are evaporated in vacuo and the resulting residue is purified by column chromatography on silica gel (DCM/MeOH/H₂O, 10:3:0.5) to afford compound 12 (8.6 mg, 66%). [α]_{D}²⁰ −39.3 (c 1.00, MeOH); ¹H NMR (500 MHz, CD₃OD): δ=0.14 (s, 9H, Si(CH₃)₃), 0.43-0.56, 0.57-0.73, 0.81-0.95, 1.06-1.45, 1.45-1.58, 1.60-1.68, 2.11-2.26 (m, 20H, C₇H₁₃^{L}, H-1, H-2, H-5, H-6a, CH₂), 0.86 (t, J=7.4 Hz, 3H, CH₃), 1.33 (d, J=6.5 Hz, 3H, H-6^{F}), 1.73 (dd, J=3.1, 13.4 Hz, 1H, H-6b), 1.95 (ddt, J=7.3, 10.5, 15.1 Hz, 1H, CH₂CH₃), 2.23 (t, J=7.2 Hz, 2H, CH₂), 3.10-3.23 (m, 2H, CH₂), 3.26 (dd, J=9.1, 10.3 Hz, 1H, H-4), 3.56-3.62 (m, 2H, H-3^{G}, H-5^{G}), 3.67-3.77 (m, 4H, H-3, H-2^{F}, H-4^{F}, H-6a^{G}), 3.77-3.84 (m, 2H, H-2^{L}, H-6b^{G}), 3.88 (dd, J=3.3, 10.3 Hz, 1H, H-3^{F}), 3.94 (m, 1H, H-4^{G}), 4.71 (d, J=8.1 Hz, 1H, H-1^{G}), 4.94 (d, J=4.0 Hz, 1H, H-1^{F}), 4.95-5.00 (m, 1H, H-5^{F}), 5.37-5.46 (m, 1H, H-2^{G}), 7.46-7.54, 7.58-7.65, 8.03-8.09 (m, 5H, C₆H₅), 7.80 (t, J=5.7 Hz, 1H, NH); ¹³C NMR (126 MHz, CD₃OD): δ=0.25 (Si(CH₃)₃), 10.75 (CH₃), 16.74 (C-6^{F}), 18.07 (CH₂), 24.56 (CH₂CH₃), 26.60, 26.79, 27.38, 27.48, 29.65, 32.89, 33.08, 34.40, 35.37, 35.57, 39.84, 43.24, 45.19 (C₇H₁₃^{L}, C-1, C-2, C-5, C-6, 2 CH₂), 63.03 (C-6^{G}), 67.63 (C-4^{G}), 67.82 (C-5^{F}), 70.31 (C-2^{F}), 71.34 (C-3^{F}), 73.00 (C-2^{G}), 73.96 (C-4^{F}), 75.98 (C-5^{G}), 80.16 (2C, C-3, C-2^{L}), 80.55 (C-4), 83.84 (C-3^{G}), 85.47 (CH₂CCSi), 100.24 (C-1^{F}), 100.63 (C-1^{G}), 107.65 (CH₂CCSi), 129.74, 130.86, 131.70, 134.28 (6C, C₆H₅), 166.87, 177.28 (3C, 3 C═O); ESI-MS: m/z: Calcd for C₄₅H₆₉NNaO₁₅Si [M+Na]⁺: 914.43, found: 914.52.

Synthesis of Compound 13:

TBAF (15 μL, 1M in THF) is added via syringe at 10-15° C. (waterbath) to a solution of compound 12 (9.0 mg, 0.010 mmol) in THF (500 μL). After 46 hours, the solvent is removed in vacuo, and the resulting residue is purified by column chromatography on silica gel (DCM/MeOH/water, 10:3:0.5) to give compound 13 (7.8 mg, 95%) as a white solid. [α]_{D}²⁰ −64.5 (c 1.08, MeOH); ¹H NMR (500 MHz, CD₃OD): δ=0.45-0.73, 0.81-0.95, 1.00-1.59, 1.59-1.69, 2.10-2.25 (m, 20H, C₇H₁₃^{L}, H-1, H-2, H-5, H-6a, CH₂), 0.86 (t, J=7.4 Hz, 3H, CH₃), 1.33 (d, J=6.5 Hz, 3H, H-6^{F}), 1.74 (m, 1H, H-6b), 1.95 (m, 1H, CH₂CH₃), 2.19 (td, J=2.7, 7.2 Hz, 2H, CH₂), 2.28 (t, J=2.7 Hz, 1H, CH), 3.12-3.29 (m, 3H, H-4, CH₂), 3.56-3.62 (m, 2H, H-3^{G}, H-5^{G}), 3.66-3.77 (m, 4H, H-3, H-2^{F}, H-4^{F}, H-6a^{G}), 3.77-3.85 (m, 2H, H-6b^{G}, H-2^{L}), 3.88 (dd, J=3.3, 10.3 Hz, 1H, H-3^{F}), 3.94 (d, J=2.9 Hz, 1H, H-4^{G}), 4.70 (d, J=8.0 Hz, 1H, H-1^{G}), 4.93 (d, J=4.0 Hz, 1H, H-1^{F}), 4.97 (q, J=6.5 Hz, 1H, H-5^{F}), 5.42 (m, 1H, H-2^{G}), 7.45-7.53, 7.58-7.64, 8.03-8.08 (m, 5H, C₆H₅), 7.79 (t, J=5.7 Hz, 1H, NH); ¹³C NMR (126 MHz, CD₃OD): δ=10.75 (CH₃), 16.70 (CH₂), 16.74 (C-6^{F}), 24.55 (CH₂CH₃), 26.58, 26.79, 27.39, 29.51, 32.93, 33.11, 34.40, 35.36, 35.52, 39.45, 43.25, 43.40, 45.24 (C₇H₁₃^{L}, C-1, C-2, C-5, C-6, 2 CH₂), 63.04 (C-6^{G}), 67.66 (C-4^{G}), 67.83 (C-5^{F}), 70.09 (CH), 70.32 (C-2^{F}), 71.35 (C-3^{F}), 73.02 (C-2^{G}), 73.97 (C-4^{F}), 76.01 (C-5^{G}), 80.17 (2C, C-3, C-2^{L}), 80.54 (C-4), 83.83 (C-3G), 84.27 (—CCH), 100.29 (C-1^{F}), 100.66 (C-1^{G}), 129.72, 130.86, 131.70, 134.31 (6C, C₆H₅), 166.85, 177.30 (3C, 3 C═O); ESI-MS: m/z: Calcd for C₄₂H₆₁NNaO₁₅ [M+Na]⁺: 842.39, found: 842.60.

Synthesis of Compound 15a:

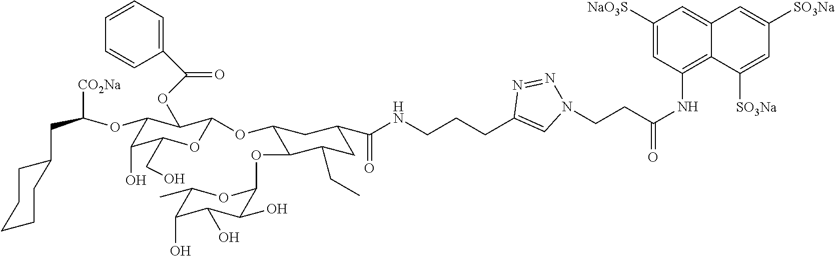

To a mixture of compound 13 (3.1 mg, 3.8 μmol) and compound 14a (3.3 mg, 5.7 μmol, example 3 below) in tert-BuOH/H₂O (1:1, 300 μL), cupric sulfate pentahydrate (0.24 mg, 1.0 μmol) and L-(+)-ascorbic acid sodium salt (0.38 mg, 1.9 μmol) are added successively. After 1 hour, the reaction mixture is purified by column chromatography on silica (DCM/MeOH/H₂O, 10:6:1.5) followed by ion-exchange chromatography (Na+ form) and RP-18 chromatography (H₂O/MeOH, 4:1) to yield compound 15a (3.2 mg, 60%) as a white solid. [α]_{D}²⁰ −25.1 (c 0.35, MeOH); ¹H NMR (500 MHz, CD₃OD): δ=0.42-0.72, 0.85-0.94, 1.04-1.42, 1.42-1.65, 1.65-1.92 (m, 23H, C₇H₁₃^{L}, CH₂CH₃, H-2a, H-5, H-6, 2 CH₂), 0.83 (t, J=7.4 Hz, 3H, CH₃), 1.28 (d, J=6.7 Hz, 3H, H-6^{F}), 1.97-2.11 (m, 2H, CH₂), 2.11-2.30 (m, 2H, H-1, H-2b), 2.55-2.69 (m, 2H, CH₂), 2.72 (t, J=7.5 Hz, 2H, CH₂), 3.08-3.28 (m, 3H, CH₂, H-4), 3.50-3.57 (m, 2H, H-3^{G}, H-5^{G}), 3.59-3.85 (m, 6H, H-3, H-2^{F}, H-4^{F}, H-6^{G}, H-2^{L}), 3.84-3.98 (m, 2H, H-4^{G}, H-3^{F}), 4.48 (t, J=6.8 Hz, 2H, CH₂), 4.69 (d, J=8.1 Hz, 1H, H-1^{G}), 4.91-4.97 (m, 2H, H-1^{F}, H-5^{F}), 5.37 (t, J=8.9 Hz, 1H, H-2^{G}), 7.40 (t, J=7.7 Hz, 2H, C₆H₅), 7.55 (tt, J=1.2, 7.3 Hz, 1H, C₆H₅), 7.96 (s, 1H, H^{T}), 8.01-8.09 (m, 2H, C₆H₅), 8.30 (d, J=1.9 Hz, 1H, H^{naph}), 8.51 (d, J=2.0 Hz, 1H, H^{naph}), 8.58 (t, J=2.5 Hz, 1H, H^{naph}), 8.99 (d, J=1.9 Hz, 1H, H^{naph}); ¹³C NMR (126 MHz, CD₃OD): δ=10.59 (CH₃), 16.67 (C-6^{F}), 23.03, 23.71, 26.55, 26.80, 27.40, 29.56, 30.19, 30.67, 33.07, 34.40, 35.40, 36.98, 37.31, 39.63, 42.95, 43.53, 50.81, 52.30 (19C, C₇H₁₃^{L}, C-1, C-2, C-5, C-6, 8 CH₂), 63.17 (C-6^{G}), 67.84 (2C, C-4^{G}, C-5^{F}), 70.34 (C-2^{F}), 71.23 (C-3^{F}), 72.98 (C-2^{G}), 73.97 (C-4^{F}), 75.73 (C-5^{G}), 80.10, 80.51 (3C, C-3, C-4, C-2^{L}), 83.63 (C-3^{G}), 99.90 (C-1^{F}), 100.64 (C-1^{G}), 101.39, 123.81, 123.95, 125.33, 128.35, 129.62, 130.89, 131.48, 131.72, 134.19, 134.55, 136.37, 142.14, 142.85, 144.34, 148.48 (19C, Ar—C), 166.96, 174.17, 177.33 (4C, 4 C═O).

Synthesis of Compound 15b:

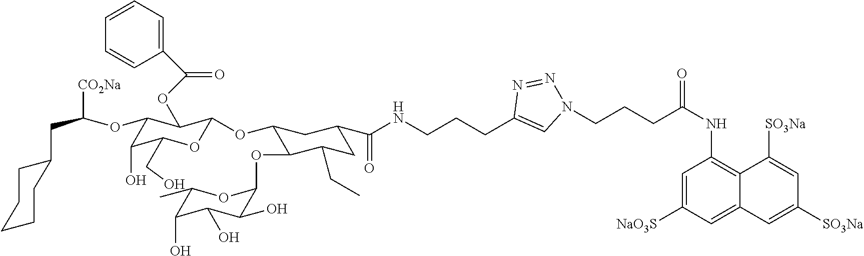

According to the procedure for compound 15a, compound 13 (3.5 mg, 4.3 μmol) and compound 14b (4.2 mg, 8.5 μmol, example 3 below) were reacted in the presence of cupric sulfate pentahydrate (0.26 mg, 1.1 μmol) and L-(+)-ascorbic acid sodium salt (0.42 mg, 2.2 μmol) to yield compound 15b (4.2 mg, 75%) as a white solid. [α]_{D}²⁰ −41.9 (c 0.42, MeOH); ¹H NMR (500 MHz, CD₃OD): δ=0.42-0.72, 0.85-0.93, 1.06-1.37, 1.37-1.42, 1.46-1.50, 1.51-1.68, 1.68-1.78, (m, 18H, C₇H₁₃^{L}, CH₂CH₃, H-2a, H-5, H-6) 0.82 (t, J=7.4 Hz, 3H, CH₃), 1.31 (d, J=6.5 Hz, 3H, H-6^{F}), 1.80-1.94 (m, 3H, CH₂CH₃, CH₂), 2.17 (d, J=12.8 Hz, 1H, H-2b), 2.23-2.34 (m, 1H, H-1), 2.38 (p, J=7.0 Hz, 2H, CH₂), 2.61 (t, J=7.2 Hz, 2H, CH₂), 2.73 (t, J=7.5 Hz, 2H, CH₂), 3.11-3.21 (m, 2H, CH₂), 3.28 (m, 1H, H-4), 3.51-3.61 (m, 2H, H-3^{G}, H-5^{G}), 3.68-3.84 (m, 6H, H-3, H-2^{F}, H-4^{F}, H-2^{L}, H-6^{G}), 3.90-3.92 (m, 1H, H-4^{G}), 3.95 (dd, J=3.3, 10.3 Hz, 1H, H-3^{F}), 4.53 (t, J=6.8 Hz, 2H, CH₂), 4.71 (d, J=8.1 Hz, 1H, H-1^{G}), 4.93-4.97 (m, 2H, H-1^{F}, H-5^{F}), 5.39 (dd, J=8.0, 9.7 Hz, 1H, H-2^{G}), 7.43 (t, J=7.8 Hz, 2H, C₆H₅), 7.52-7.61 (m, 1H, C₆H₅), 7.99 (s, 1H, H^{T}), 8.05 (dd, J=1.4, 7.7 Hz, 2H, C₆H₅), 8.30 (d, J=1.9 Hz, 1H, H^{naph}), 8.50 (d, J=2.0 Hz, 1H, H^{naph}), 8.56 (d, J=1.9 Hz, 1H, H^{naph}), 8.99 (d, J=1.9 Hz, 1H, H^{naph}); ¹³C NMR (126 MHz, CD₃OD): δ=10.58 (CH₃), 16.67 (C-6^{F}), 23.65, 24.73, 26.56, 26.71, 26.80, 27.42, 30.12, 33.06, 34.28, 34.43, 35.42, 35.52, 39.58, 43.02, 43.57, 44.89, 50.74 (18C, C₇H₁₃^{L}, 7 CH₂, C-1, C-2, C-5, C-6), 63.19 (C-6^{G}), 67.72, 67.87 (C-4^{G}, C-5^{F}), 70.33 (C-2^{F}), 71.22 (C-3^{F}), 72.98 (C-2^{G}), 73.96 (C-4^{F}), 75.85 (C-5^{G}), 80.04, 80.10, 80.54 (C-3, C-4, C-2^{L}), 83.68 (C-3^{G}), 99.85, 100.67 (C-1^{F}, C-1^{G}, C^{T}), 124.05, 124.07, 125.33, 125.43, 128.34, 129.64, 130.88, 131.43, 131.72, 134.20, 134.41, 136.36, 142.09, 142.89, 144.38, 148.44 (18C, Ar—C), 166.95, 173.24, 177.34, 183.23 (4 C═O).

Synthesis of Compound 15c:

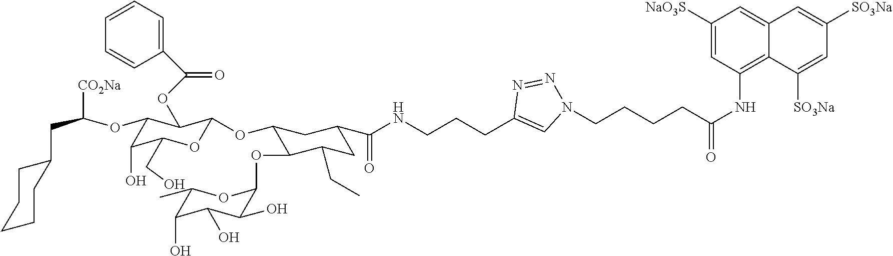

According to the procedure for compound 15a, compound 13 (3.5 mg, 4.3 μmol) and compound 14c (3.5 mg, 6.4 μmol, example 3 below) were reacted in the presence of cupric sulfate pentahydrate (0.26 mg, 1.1 μmol) and L-(+)-ascorbic acid sodium salt (0.42 mg, 2.2 μmol) to yield compound 15c (4.2 mg, 70%) as a white solid. [α]_{D}²⁰ −23.9 (c 0.46, MeOH); ¹H NMR (500 MHz, D₂O): δ=0.19-0.34, 0.38-0.50, 0.54-0.77, 1.31-1.43, 1.44-1.55, 1.65-1.77 (m, 20H, C₇H_{D}^{L}, CH₂, CH₂CH₃, H-2a, H-5, H-6), 0.82 (t, J=7.4 Hz, 3H, CH₃), 1.30 (d, J=6.6 Hz, 3H, H-6^{F}), 1.76-1.87 (m, 1H, CH₂CH₃), 2.09-2.16 (m, 1H, H-2b), 2.16-2.26 (m, 1H, H-1), 2.67 (t, J=7.5 Hz, 2H, CH₂), 2.91-3.05 (m, 2H, CH₂), 3.17-3.27 (m, 3H, CH₂, H-4), 3.72 (td, J=4.5, 9.3, 10.2 Hz, 1H, H-3), 3.76-3.88 (m, 7H, H-2^{L}, H-2^{F}, H-4^{F}, H-3^{G}, H-5^{G}, H-6^{G}), 3.92 (dd, J=3.4, 10.5 Hz, 1H, H-3^{F}), 4.00 (d, J=3.2 Hz, 1H, H-4^{G}), 4.86-4.94 (m, 3H, CH₂, H-1^{G}), 4.96-5.04 (m, 2H, H-1^{F}, H-5^{F}), 5.15-5.29 (m, 1H, H-2^{G}), 7.28 (t, J=7.7 Hz, 2H, C₆H₅), 7.46 (t, J=7.3 Hz, 1H, C₆H₅), 7.94-7.99 (m, 3H, C₆H₅, H^{T}), 8.05 (d, J=2.0 Hz, 1H, H^{naph}), 8.49 (d, J=2.0 Hz, 1H, H^{naph}), 8.64 (d, J=2.0 Hz, 1H, H^{naph}), 8.76 (d, J=1.9 Hz, 1H, H^{naph}); ¹³C NMR (126 MHz, D₂O): 6=9.48 (CH₃), 15.39 (C-6^{F}), 21.95 (CH₂), 24.83, 25.17, 25.59, 28.42, 30.64, 31.20, 32.87, 33.51, 34.68, 37.34, 38.44, 41.30, 42.85 (15C, C₇H₁₃^{L}, C-1, C-2, C-5, C-6, 4 CH₂), 46.49 (CH₂), 61.75 (C-6^{G}), 66.13 (C-4^{G}), 66.63 (C-5^{F}), 68.27 (C-2^{F}), 69.29 (C-3^{F}), 72.08, 72.26 (C-4^{F}, C-2^{G}), 74.22 (C-5^{G}), 78.88, 79.35 (C-3, C-2^{L}), 80.12 (C-4), 80.98 (C-3^{G}), 98.71 (C^{T}), 99.17 (C-1^{F}), 99.99 (C-1^{G}), 123.67, 124.75, 125.32, 126.38, 126.98, 128.63, 128.72, 129.68, 131.38, 131.52, 134.00, 135.11, 139.03, 139.79, 141.30, 147.69 (18C, Ar—C), 167.99, 171.70, 176.98, 182.71 (4 C═O).

### Example 2

### Synthesis of Seletin Modulator 17 (FIG. 2)

Synthesis of Compound 17:

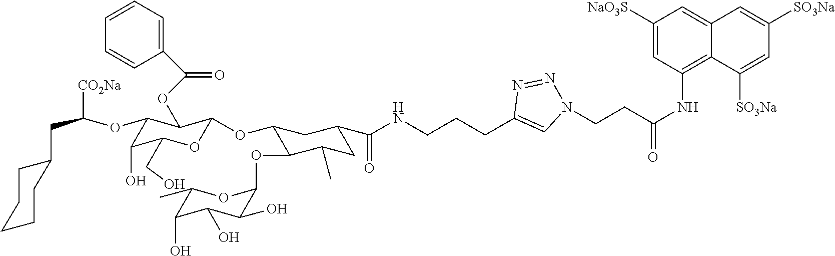

According to the procedure for compound 15a, compound 16 (3.3 mg, 4.1 μmol, synthesized as described by Egger et al., J. Am. Chem. Soc. 135:9820-9828, 2013) and compound 14a (3.4 mg, 6.2 μmol) in the presence of cupric sulfate pentahydrate (0.25 mg, 1.0 μmol) and L-(+)-ascorbic acid sodium salt (0.40 mg, 2.1 μmol) to yield compound 17 (3.2 mg, 57%) as a white solid. [α]_{D}²⁰ −16.5 (c 0.25, MeOH); ¹H NMR (500 MHz, D₂O): 6=0.19-0.33, 0.37-0.49, 0.54-0.76, 0.92-1.04, 1.07-1.23, 1.31-1.43 (15H, C₇H_{D}^{L}, H-2a, H-6a), 1.06 (d, J=6.5 Hz, 3H, CH₃), 1.30 (d, J=6.5 Hz, 3H, H-6^{F}), 1.55 (d, J=13.9 Hz, 1H, H-6b), 1.62 (m, 1H, H-5), 1.70 (tt, J=4.3, 7.6 Hz, 2H, CH₂), 2.11 (m, 1H, H-2b), 2.24 (ddd, J=3.8, 12.7, 16.5 Hz, 1H, H-1), 2.67 (dd, J=6.9, 8.3 Hz, 2H, CH₂), 2.90-3.03 (m, 2H, CH₂), 3.12 (t, J=9.7 Hz, 1H, H-4), 3.20 (dd, J=5.3, 7.4 Hz, 2H, CH₂), 3.71 (ddd, J=4.7, 9.3, 11.4 Hz, 1H, H-3), 3.77-3.85 (m, 6H, H-3^{G}, H-5^{G}, H-6^{G}, H-2^{F}, H-2^{L}), 3.86 (dd, J=1.1, 3.5 Hz, 1H, H-4^{F}), 3.92 (dd, J=3.4, 10.5 Hz, 1H, H-3^{F}), 4.00 (d, J=3.2 Hz, 1H, H-4^{G}), 4.87 (d, J=8.0 Hz, 1H, H-1^{G}), 4.88-4.93 (m, 2H, CH₂), 4.99 (m, 1H, H-5^{F}), 5.05 (d, J=4.1 Hz, 1H, H-1^{F}), 5.21 (dd, J=8.0, 9.7 Hz, 1H, H-2^{G}), 7.28 (t, J=7.9 Hz, 2H, C₆H₅), 7.46 (m, 1H, C₆H₅), 7.88-8.02 (m, 3H, C₆H₅, H^{T}), 8.05 (d, J=1.9 Hz, 1H, H^{naph}), 8.49 (d, J=2.0 Hz, 1H, H^{naph}), 8.65 (d, J=2.0 Hz, 1H, H^{naph}), 8.76 (d, J=1.9 Hz, 1H, H^{naph}); ¹³C NMR (126 MHz, D₂O): δ=16.38 (C-6^{F}), 18.71 (CH₃), 22.95 (CH₂), 25.84, 26.18, 26.60, 29.41, 32.21, 33.88, 34.53, 35.72, 35.97, 38.01, 38.35, 39.43, 42.35, 42.54 (C₇H₁₃^{L}, 3 CH₂, C-1, C-2, C-5, C6), 47.50 (CH₂), 62.77 (C-6^{G}), 67.14 (C-4^{G}), 67.57 (C-5^{F}), 69.26 (C-2^{F}), 70.35 (C-3^{F}), 73.10, 73.29 (C-2^{G}, C-4^{F}), 75.23 (C-5^{G}), 79.89 (C-3), 80.09 (C-2^{L}), 81.98 (C-3^{G}), 83.58 (C-4), 99.85, 100.24 (3C, C-1^{F}, C-1^{G}, C^{T}), 124.68, 125.76, 126.32, 127.38, 127.99, 129.64, 129.73, 130.69, 132.38, 132.53, 136.12, 140.04, 140.80, 142.30, 148.69 (17C, Ar—C), 169.00, 172.70, 177.83, 183.72 (4 C═O).

### Example 3

### Synthesis of Compounds 14a-c (FIG. 3)

Synthesis of Compound 18:

A mixture of 3-chloropropanoic acid (150 mg, 1.38 mmol) and NaN₃ (898 mg, 13.8 mmol) in water (3 mL) is stirred at reflux. After 22 hours, the reaction mixture is cooled to room temperature, acidified with aqueous HCl and extracted with diethyl ether. The organic layer is dried with Na₂SO₄ and evaporated to dryness to give 3-azidopropanoic acid, compound 18, (130 mg, 81%).

Synthesis of Compound 14a:

To a solution of compound 18 (16.0 mg, 0.139 mmol) in DMF (300 DIPEA (40 μL) and COMU (71 mg, 0.167 mmol) are added successively at 0° C. After 5 minutes, a pre-cooled (0° C.) mixture of sodium 8-aminonaphthalene-1,3,6-trisulfonate (19) (62.0 mg, 0.146 mmol) and DIPEA (40 μL) in DMF (300 μL) is added dropwise. After 1 hour at 0° C., the reaction mixture is stirred for additional 21 hours at room temperature. Evaporation of the solvent and purification by chromatography on silica gel (DCM/MeOH/water, 10:5:1) yields compound 14a (48 mg, 64%) as a DIPEA complex. NMR (500 MHz, CD₃OD): δ=1.25-1.33 (m, 30H, 10 CH₃^{DIPEA}), 2.85 (t, J=6.7 Hz, 2H, CH₂), 3.12 (q, J=7.4 Hz, 4H, 2 CH₂^{DIPEA}), 3.63 (h, J=6.6 Hz, 4H, 4 CH^{DIPEA}), 3.72 (t, J=6.7 Hz, 2H), 8.28 (d, J=1.9 Hz, 1H, H^{naph}), 8.48 (d, J=2.0 Hz, 1H, H^{naph}), 8.57 (d, J=1.9 Hz, 1H, H^{naph}), 8.95 (d, J=2.0 Hz, 1H, H^{naph}), 11.74 (s, 1H, NH); ¹³C NMR (126 MHz, CD₃OD): δ=11.81, 15.89, 17.35 (10C, CH₃^{DIPEA}), 35.95 (CH₂, 42.45 (CH₂^{DIPEA}), 47.04 (CH^{DIPEA}), 54.47 (CH₂), 122.70, 123.88, 123.91, 126.96, 129.81, 133.14, 134.91, 140.87, 141.74, 143.24 (10C, C^{naph}), 170.23 (C═O).

Synthesis of Compound 20:

To a solution of 4-chlorobutyric acid (17.6 μL, 0.178 mmol) in DMF (500 μL) at 0° C., DIPEA (93.0 μL, 0.535 mmol) and COMU (91.0 mg, 0.214 mmol) are added successively. After 5 minutes, a pre-cooled (0° C.) mixture of compound 19 (80.0 mg, 0.187 mmol) and DIPEA (93.0 μL, 0.535 mmol) in DMF (500 μL) is added dropwise. After 1 hour at 0° C., stirring is continued for additional 65 hours at room temperature. Evaporation of the solvent and purification of the residue by chromatography on silica gel (DCM/MeOH/water, 10:6:1.2) yields compound 20 (41 mg, 48%) as a di-DIPEA complex. NMR (500 MHz, CD₃OD): δ=1.26-1.33 (m, 30H, 10 CH₃^{DIPEA}), 2.23 (h, J=6.3, 7.0 Hz, 2H, CH₂), 2.73 (t, J=7.4 Hz, 2H, CH₂), 3.13 (q, J=7.4 Hz, 4H, 2 CH₂^{DIPEA}), 3.63 (p, J=6.6 Hz, 4H, 4 CH^{DIPEA}), 3.70 (t, J=6.7 Hz, 2H, CH₂), 8.28 (d, J=1.9 Hz 1H, H^{naph}), 8.47 (d, J=1.9 Hz, 1H, H^{naph}), 8.54 (d, J=1.9 Hz, 1H, H^{naph}), 8.95 (d, J=1.9 Hz, 1H, H^{naph}), 11.66 (s, 1H, NH); ¹³C NMR (126 MHz, CD₃OD): δ=13.24, 17.32, 18.78 (10C, CH₃^{DIPEA}), 29.71, 35.26 (CH₂), 43.87 (CH₂^{DIPEA}), 45.33 (CH₂), 55.89 (CH₂^{DIPEA}), 124.22, 125.30, 125.39, 128.34, 131.29, 134.70, 136.36, 142.26, 143.07, 144.58 (10C, C^{naph}), 173.74 (C═O).

Synthesis of Compound 14b:

A mixture of compound 20 (41 mg, 0.084 mmol) and NaN₃ (54 mg, 0.84 mmol) in water (1.5 mL) is stirred at reflux. After 18 hours, the reaction is cooled and lyophilized. Purification by chromatography on RP-18 (H₂O/MeOH, 4:1) gives compound 14b (12 mg, 29%) as a di-DIPEA complex. ¹H NMR (500 MHz, D₂O): δ=1.23-1.35 (m, 30H, 10 CH₃^{DIPEA}), 2.03 (p, J=6.9 Hz, 2H, CH₂), 2.68 (t, J=7.6 Hz, 2H, CH₂), 3.13 (q, J=7.4 Hz, 4H, 2 CH₂^{DIPEA}), 3.64 (h, J=6.6 Hz, 4H, 4 CH^{DIPEA}), 3.75 (t, J=6.5 Hz, 2H, CH₂), 8.24 (d, J=1.9 Hz, 1H, H^{naph}), 8.50 (d, J=2.0 Hz, 1H, H^{naph}), 8.69 (d, J=2.0 Hz, 1H, H^{naph}), 8.80 (d, J=2.0 Hz, 1H, H^{naph}); ¹³C NMR (126 MHz, D₂O): 6=12.08, 16.18, 17.66 (10C, CH₃^{DIPEA}), 27.38, 33.10 (2 CH₂), 42.49 (CH₂^{DIPEA}), 54.32 (CH^{DIPEA}), 61.06 (CH₂), 125.12, 125.66, 126.24, 126.98, 131.66, 132.03, 135.10, 139.07, 139.61, 141.11 (10C, C^{naph}), 175.60 (C═O).

Synthesis of Compound 14c:

To a solution of 5-azidopentanoic acid (60.0 mg, 0.419 mmol) in dry DCM (500 μL), 1-chloro-N,N,2-trimethylpropenylamine (67.0 μL, 0.475 mmol) is added via syringe at room temperature under argon. After 5 hours, the reaction mixture is added to a pre-stirred mixture of compound 19 (119 mg, 0.279 mmol) and DMAP (10 mg) in pyridine (750 μL) at room temperature. After 65 hours, the solvents are removed in vacuo. Purification of the residue by chromatography on silica gel (DCM/MeOH/water, 10:6:1.2) yields compound 14c (42 mg, 26%) as a white solid. ¹H NMR (500 MHz, CD₃OD): δ=1.73 (dq, J=6.8, 9.9 Hz, 2H, CH₂), 1.86 (dq, J=6.1, 7.7, 8.7 Hz, 2H, CH₂), 2.62 (t, J=7.5 Hz, 2H, CH₂), 3.38 (t, J=6.8 Hz, 2H, CH₂), 8.30 (d, J=1.9, 1H Hz, H^{naph}), 8.50 (d, J=1.9 Hz, 1H, H^{naph}), 8.52 (d, J=1.8 Hz, 1H, H^{naph}), 8.95 (d, J=1.9 Hz, 1H, H^{naph}); ¹³C NMR (126 MHz, CD₃OD): δ=23.66, 29.54, 37.31, 52.29 (4 CH₂), 124.13, 125.51, 128.33, 131.51, 134.40, 136.32, 141.98 (10C, C^{naph}).

### Example 4

### Affinity Assay for E-Selectin Antagonist

For E-selectin, a construct consisting of the lectin domain, the EGF like domain, and the first two short consensus repeats (E-selectinLEC2) was labeled using the amine reactive protein labeling kit BLUE-NHS from NanoTemper Technologies GmbH (Munich, Germany).

The Microscale Thermophoresis (MST) experiments were carried out in a HEPES buffer adjusted to pH 7.4 containing 150 mM NaCl, 1 mM CaCl₂ and 0.05% Tween 20 at a temperature of 25° C. with 50% LED power, 50% laser power, laser on time of 30 seconds, and laser off time of 5 seconds with standard treated capillaries. A fixed concentration of the labeled E-selectin_{LEC2} protein was incubated for 20 minutes with a 15-datapoint linear 1:1 dilution of ligand starting about 30 times above the expected K_{D}. The observed data points were fitted using the single-site binding function of Alan Cooper, Tutorial Chemistry Texts, Vol 16: Biophysical Chemistry; Royal Society of Chemistry: Cambridge, 2004.

Binding Affinity (K_{D}) of sLe^{x} and Test Compounds
to E-Selectin Determined by MST

| | Compound | | | | |
|---|---|---|---|---|---|
| | sLe^{x} | 15a | 15b | 15c | 17 |
| K_{D} | 687 μM | 1.5 μM | 0.97 μM | 1.2 μM | 3 μM |

### Example 5

### Affinity Assay for P-Selectin Antagonists

The cell-free P-selectin ligand binding assay was adapted from Weitz-Schmidt et al (G. Weitz-Schmidt, D. Stokmaier, G. Scheel, N. E. Nifant'ev, A. B. Tuzikov, N. V. Bovin, Anal Biochem 1996, 238, 184-190). A MaxiSorp 96-well flat bottom microtiter plate purchased from Nalge Nunc International (Penfield, N.Y., USA) was coated with P-selectin/IgG by incubation with 100 μL/well of the purified protein at a concentration of 3 μg/ml in 10 mM HEPES-NaOH pH 7.4, 150 mM NaCl, 1 mM CaCl₂ (HAB-Ca⁺). After an overnight incubation at 4° C., the plate was washed three times with HAB-Ca⁺ followed by a 2 hour incubation with 200 μL/well of a solution of 3% BSA in HAB-Ca⁺ at room temperature to block nonspecific binding. During this incubation, inhibitory test compounds, diluted in HAB-Ca⁺, were titrated by a twofold serial and mixed with an equal volume of 0.2 μg/ml of a preformed complex of a biotinylated sialyl Lewis' polyacrylamide polymer (sLe^{a}-PAA) purchased form GlycoTech Inc. (Gaithsburg, Md., USA) and horseradish peroxidase-labeled streptavidin (streptavidin-POD) purchased from Roche (Rotkreuz, Switzerland). After blocking, the microtiter plate was washed three times with HAB-Ca⁺ and 100 μL/well of the inhibitor dilution series premixed with sLe^{a}-PAA streptavidin-POD complex was transferred to the different wells of the plate. The binding reaction was allowed to proceed at 25° C. After 2 hours, the plate was washed twice with HAB-Ca⁺, and 100 μL/well of ABTS (2,2′-azino-bis[3-ethylbenzthiazoline-6-sulfonic acid]) substrate reagent purchased from Invitrogen (Paisley, UK) was added to each well. Adding 100 μL/well of 2% oxalic acid stopped the colorimetric reaction was after 5 minutes. The optical density was determined at 450 nm (SpectraMax). The concentration of antagonist required to inhibit binding by 50% was determined and is reported as the IC₅₀ value.

Binding Affinity (IC) sLe^{x} and Test Compounds to P-Selectin
Determined by Competitive Binding Assay

| | Compound | | | | |
|---|---|---|---|---|---|
| | sLe^{x} | 15a | 15b | 15c | 17 |
| IC₅₀ | >7 mM | 94 μM | 82 μM | 210 μM | 185 μM |

The various embodiments described above can be combined to provide further embodiments. All U.S. patents, U.S. patent application publications, U.S. patent applications, non-U.S. patents, non-U.S. patent applications, and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

1. A compound of Formula (Ia): 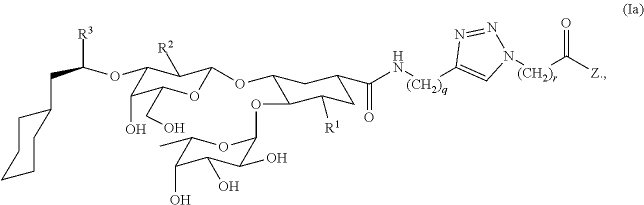
or pharmaceutically acceptable salts thereof,
wherein:
R¹ is selected from the group consisting of H, C₁₋₈ alkyl, C₂₋₈ alkenyl, C₂₋₈ alkynyl, C₄₋₁₆ cycloalkylalkyl, 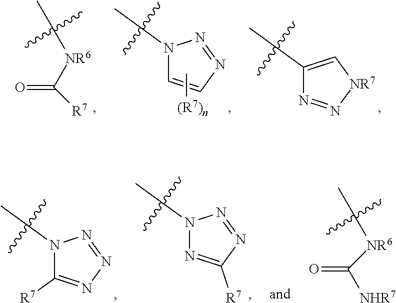
groups;
R² is selected from the group consisting of C₁₋₈ alkyl, C₂₋₈ alkenyl, C₂₋₈ alkynyl, C₄₋₁₆ cycloalkylalkyl, —OH, —OX¹, halo, —NH₂, —OC(═O)X¹, —NHC(═O)X¹, and —NHC(═O)NHX¹, wherein X¹ is selected from the group consisting of C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl;
R³ is selected from the group consisting of —CN, —CH₂CN, and —C(═O)X², wherein X² is selected from the group consisting of C₁₋₈ alkyl, C₂₋₈ alkenyl, C₂₋₈ alkynyl, —OY², —NHOH, —NHOCH₃, —NHCN, and —NY²Y³, wherein Y² and Y³ are independently selected from the group consisting of H, C₁₋₈ alkyl, C₂₋₈ alkenyl, C₂₋₈ alkynyl, and C₄₋₁₆ cycloalkylalkyl, wherein optionally Y² and Y³ are joined together to form a ring;
R⁶ is selected from the group consisting of H, C₁₋₈ alkyl, C₂₋₈ alkenyl, C₂₋₈ alkynyl, C₄₋₁₆ cycloalkylalkyl, and —C(═O)R⁷;
each R⁷ is independently selected from the group consisting of H, C₁₋₈ alkyl, C₂₋₈ alkenyl, C₂₋₈ alkynyl, C₄₋₁₆ cycloalkylalkyl, 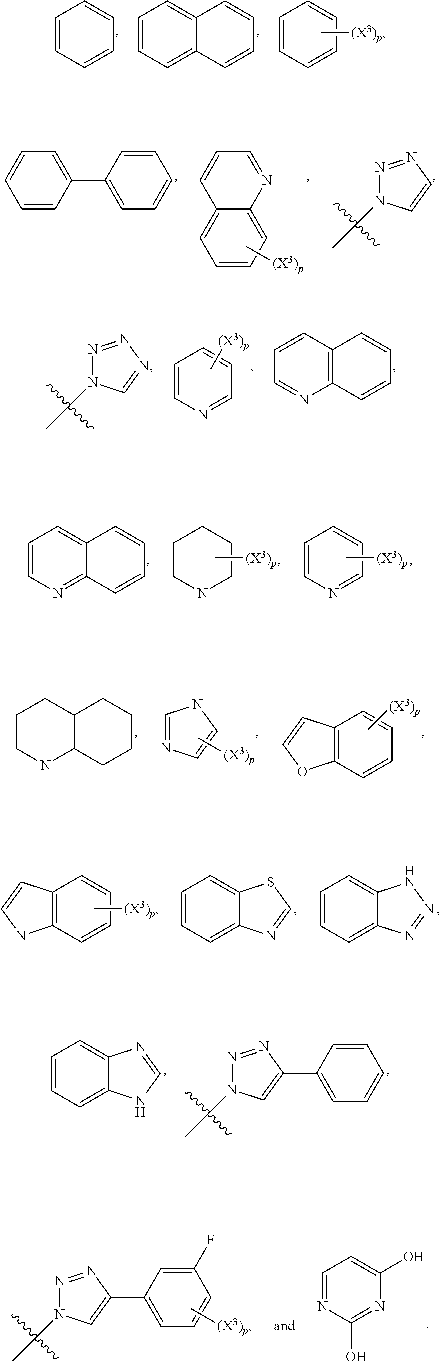
wherein each X³ is independently selected from the group consisting of H, —OH, Cl, F, N₃, —NH₂, C₁₋₈ alkyl, C₂₋₈ alkenyl, alkynyl, C₆₋₁₄ aryl, —OC₁₋₈ alkyl, —OC₂₋₈ alkenyl,
—OC₂₋₈ alkynyl, and —OC₆₋₁₄ aryl, further wherein any one to three positions of any cyclic R⁷ group is optionally substituted with a group independently selected from the group consisting of Cl, F, C₁₋₈ alkyl, C₂₋₈ alkenyl, alkynyl, C₆₋₁₄ aryl, and —OY⁴, wherein Y⁴ is selected from the group consisting of H, C₁₋₈ alkyl, Cₘ alkenyl, Cₘalkynyl, and C₆₄₄ aryl;
n is chosen from integers ranging from 0 to 2;
p is chosen from integers ranging from 0 to 3;
q is chosen from integers ranging from 1 to 10;
r is chosen from integers ranging from 1 to 10; and
Z is a benzyl amino sulfonic acid (BASA).

2. The compound according to claim 1, wherein R¹ is selected from the group consisting of methyl, ethyl,

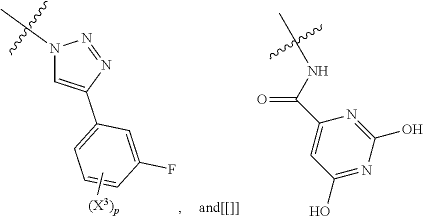

3. (canceled)

4. (canceled)

5. (canceled)

6. (canceled)

7. The compound according to claim 1, wherein R² is selected from the group consisting of —OC(═O)X¹, —NHC(═O)X¹, and —NHC(═O)NHX¹, and wherein X¹ is selected from the group consisting of C₁₋₈ alkyl, C₂₋₁₂ heterocyclyl, C₆₋₁₈ aryl, and C₁₋₁₃ heteroaryl groups.

8. The compound according to claim 1, wherein R² is selected from the group consisting of

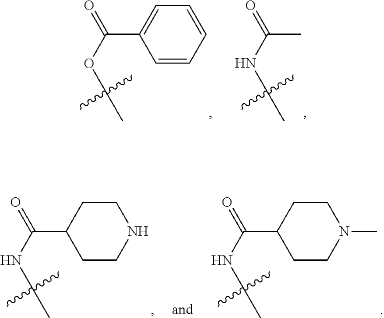

9. (canceled)

10. The compound according to claim 1, wherein R³ is —C(═O)X², wherein X² is selected from the group consisting of —OY², —NHOH, —NHOCH₃, and —NY²Y³ groups, wherein Y² and Y³ are independently selected from the group consisting of H and C₁₋₈ alkyl, and optionally wherein Y² and Y³ are joined together to form a ring.

11. The compound according to claim 10, wherein R³ is —C(═O)OH.

12. (canceled)

13. (canceled)

14. (canceled)

15. (canceled)

16. (canceled)

17. (canceled)

18. The compound according to claim 1, wherein Z is selected from the group consisting of 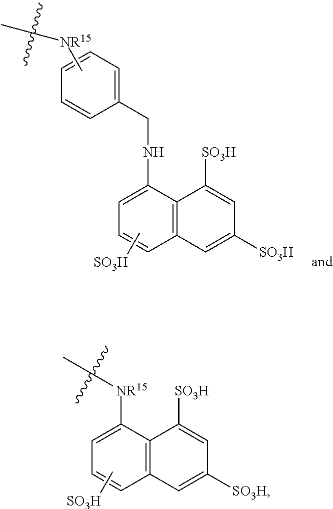
wherein R¹⁵ is selected from the group consisting of H, C₁₋₈ alkyl, —C(═O)X⁵, and —C(═O)NHX⁵, wherein X⁵ is selected from the group consisting of C₁₋₈ alkyl, C₂₋₈ alkenyl, C₂₋₈ alkynyl, C₆₋₁₈ aryl, and C₁₋₁₃ heteroaryl.

19. The compound according to claim 1, wherein Z is selected from the group consisting of

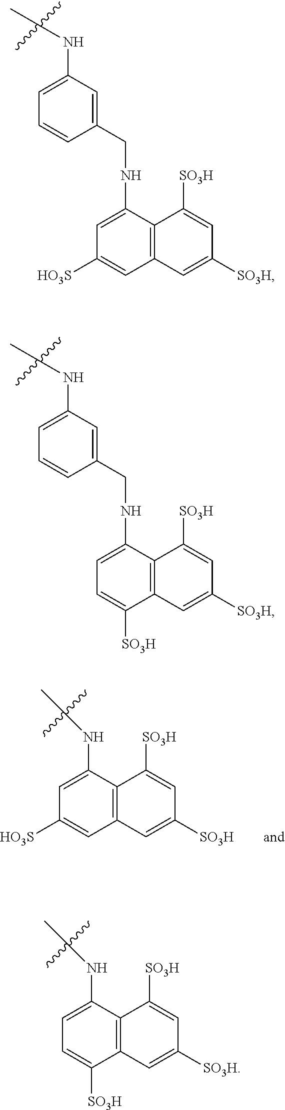 [SMILES: *Nc1cccc(CNc2cc(S(=O)(=O)O)cc3cc(S(=O)(=O)O)cc(S(=O)(=O)O)c23)c1 ; *Nc1cccc(CNc2ccc(S(=O)(=O)O)c3cc(S(=O)(=O)O)cc(S(=O)(=O)O)c23)c1 ; *Nc1cc(S(=O)(=O)O)cc2cc(S(=O)(=O)O)cc(S(=O)(=O)O)c12 ; "and" ; *Nc1ccc(S(=O)(=O)O)c2cc(S(=O)(=O)O)cc(S(=O)(=O)O)c12]

20. The compound according to claim 1, wherein the compound is selected from the group consisting of 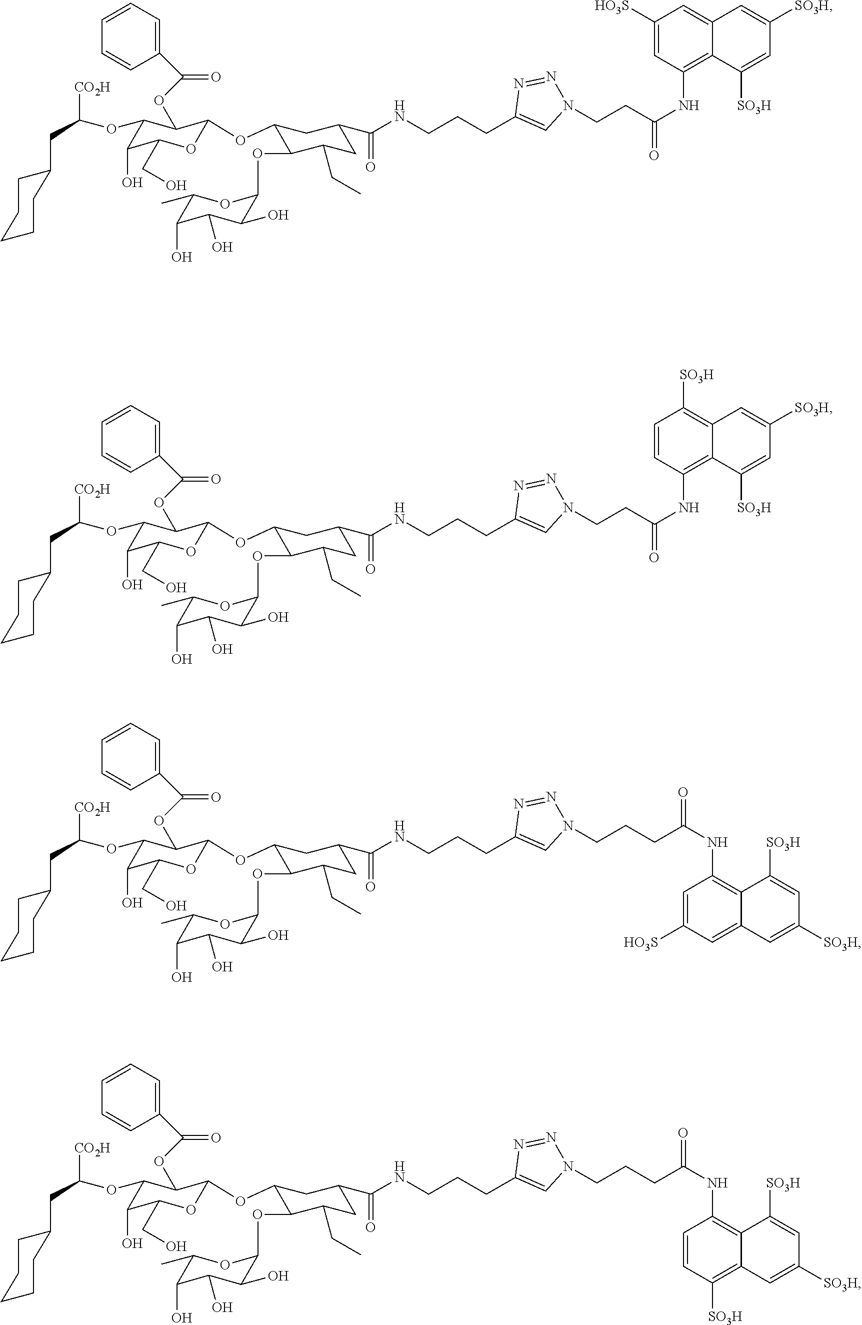 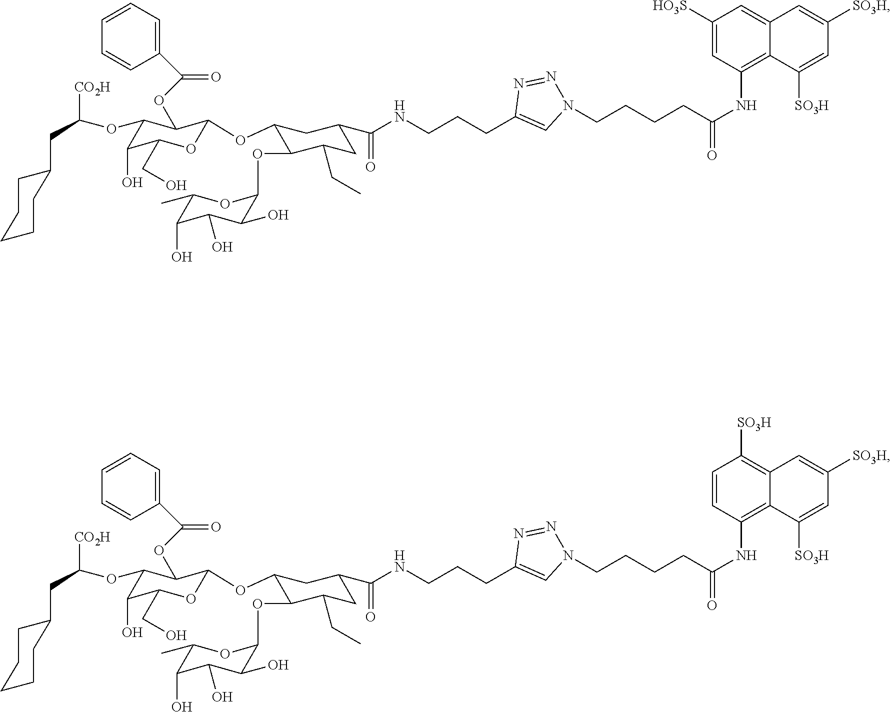 [SMILES: CCC1CC(C(=O)NCCCc2cn(CCCCC(=O)Nc3cc(S(=O)(=O)O)cc4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1OC(C)C(O)C(O)C1O ; CCC1CC(C(=O)NCCCc2cn(CCCCC(=O)Nc3ccc(S(=O)(=O)O)c4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1OC(C)C(O)C(O)C1O] 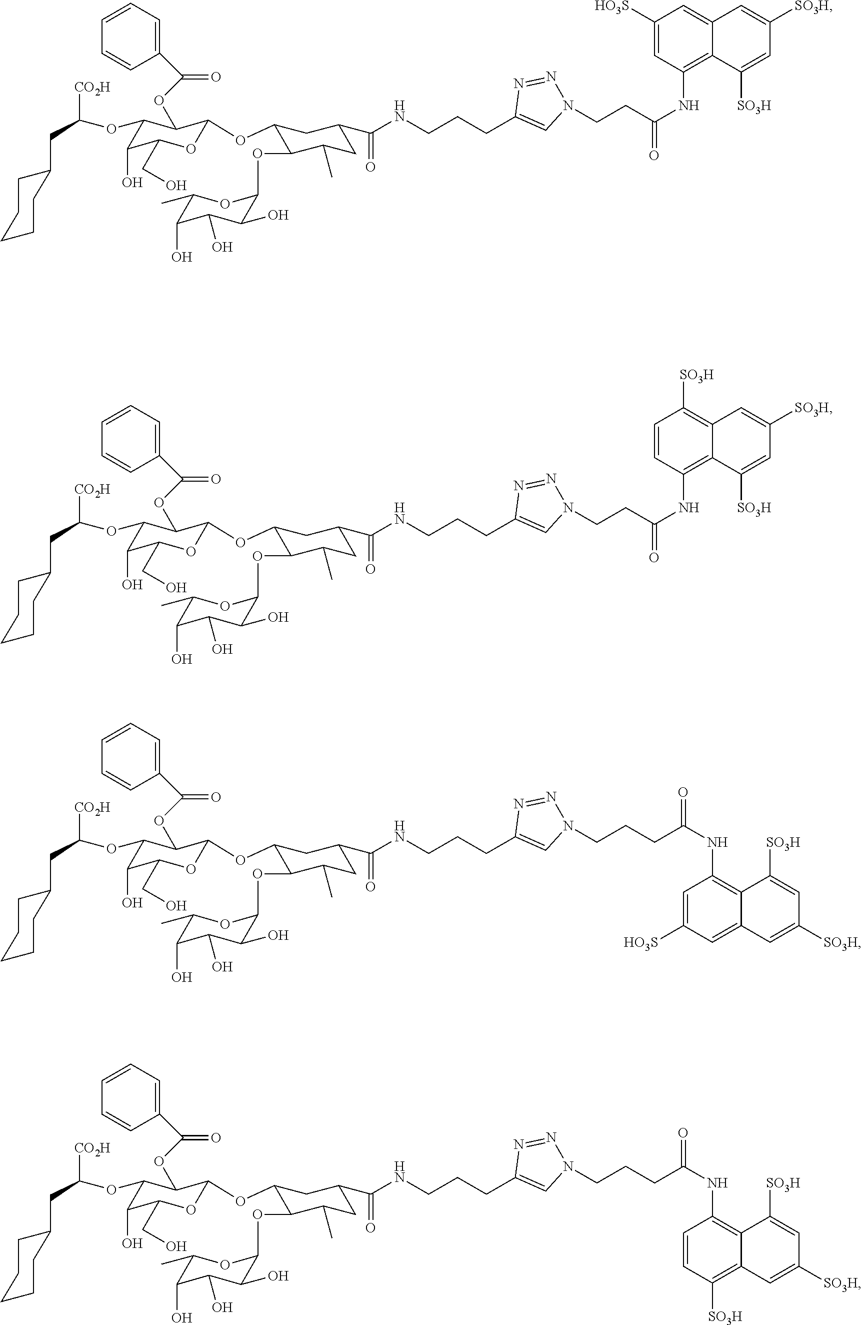 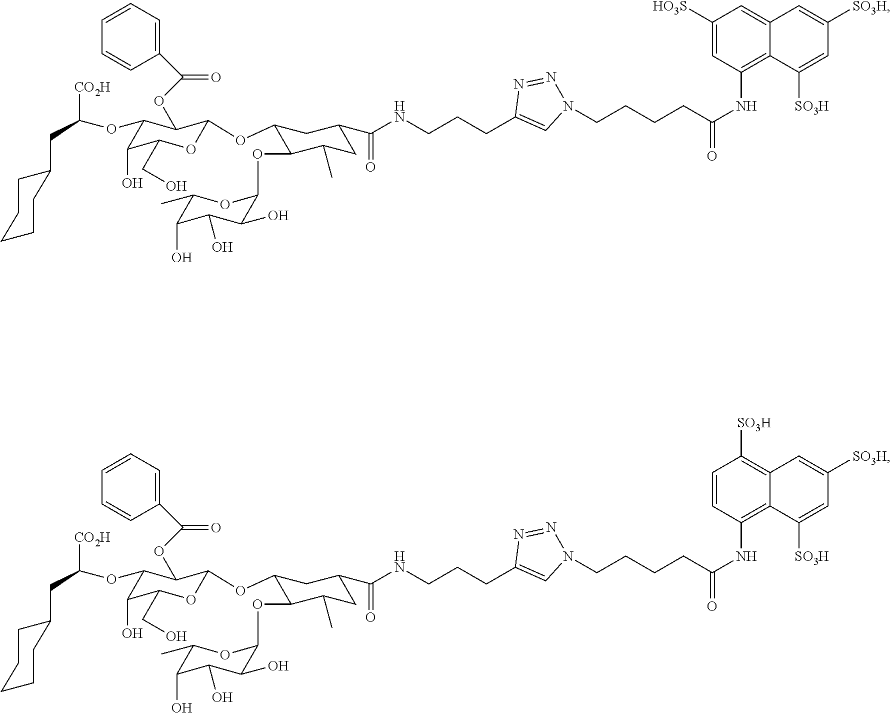 [SMILES: CC1CC(C(=O)NCCCc2cn(CCCCC(=O)Nc3cc(S(=O)(=O)O)cc4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1OC(C)C(O)C(O)C1O ; CC1CC(C(=O)NCCCc2cn(CCCCC(=O)Nc3ccc(S(=O)(=O)O)c4cc(S(=O)(=O)O)cc(S(=O)(=O)O)c34)nn2)CC(OC2OC(CO)C(O)C(O[C@@H](CC3CCCCC3)C(=O)O)C2OC(=O)c2ccccc2)C1OC1OC(C)C(O)C(O)C1O]
and pharmaceutically acceptable salts thereof.

21. (canceled)

22. The compound according to claim 1, wherein the compound is selected from the group consisting of 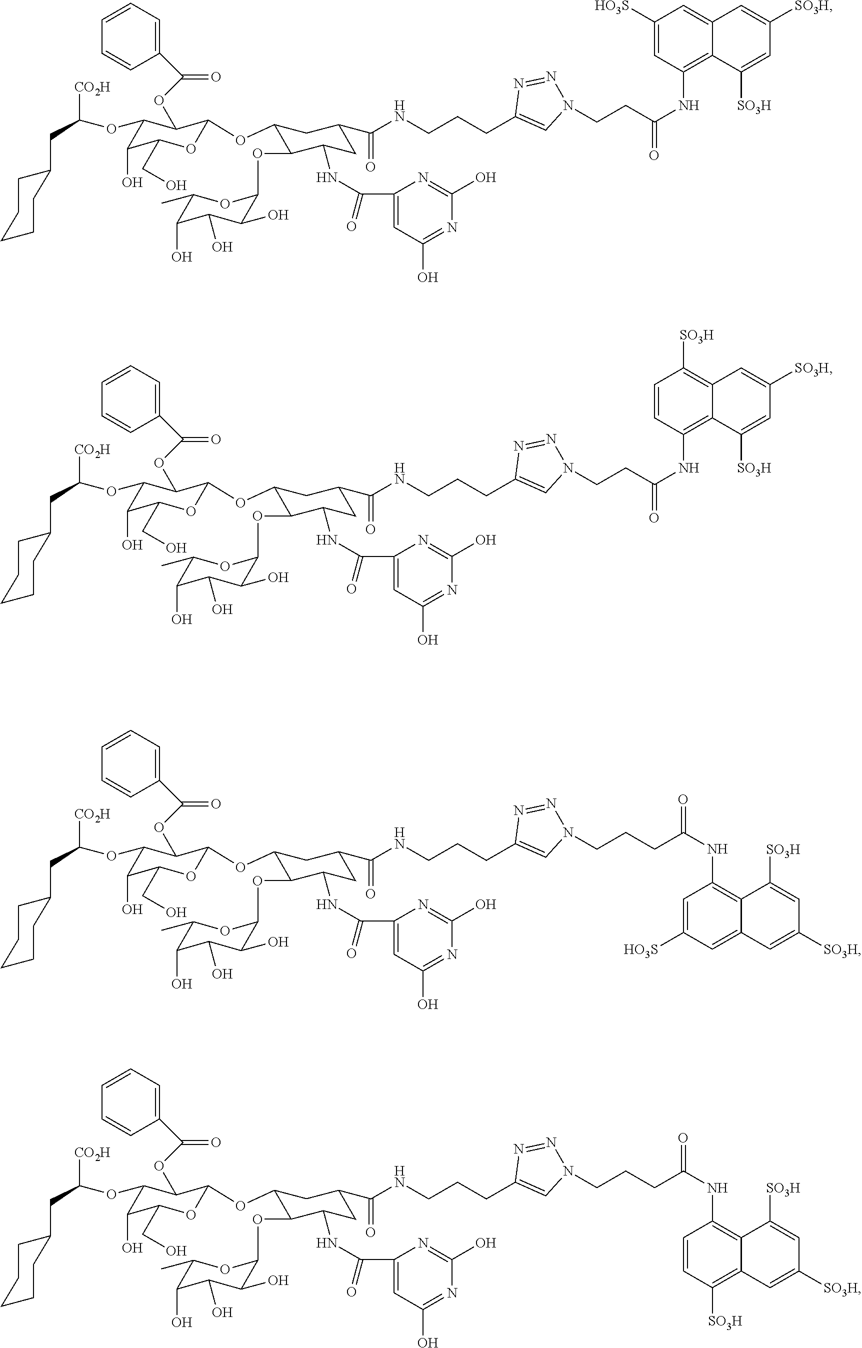 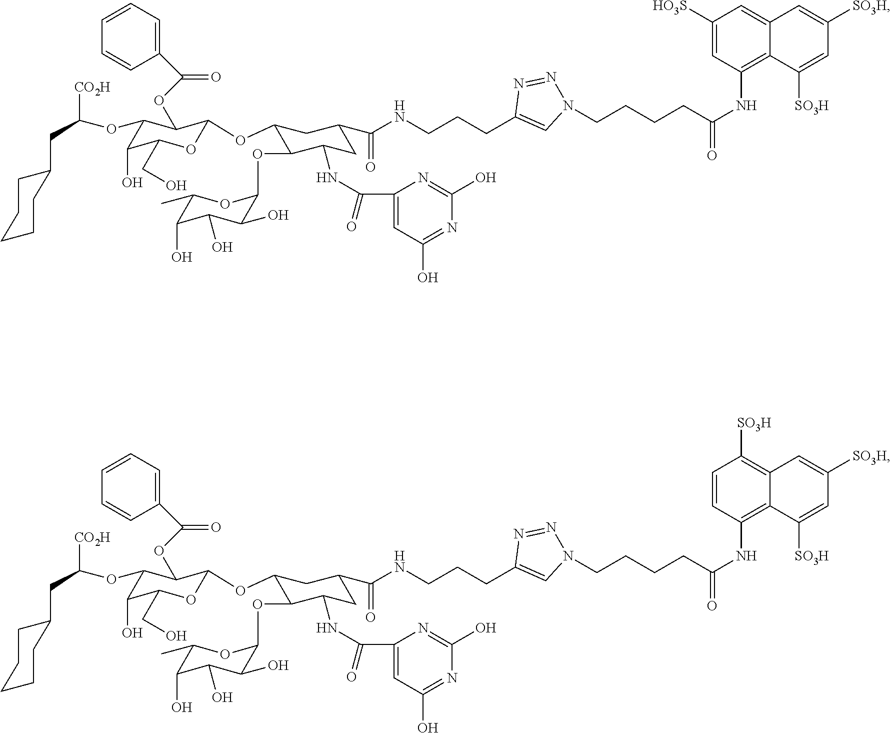 [SMILES: CC1OC(OC2C(NC(=O)c3cc(O)nc(O)n3)CC(C(=O)NCCCc3cn(CCCCC(=O)Nc4cc(S(=O)(=O)O)cc5cc(S(=O)(=O)O)cc(S(=O)(=O)O)c45)nn3)CC2OC2OC(CO)C(O)C(O[C@@H](CC3CCCCC3)C(=O)O)C2OC(=O)c2ccccc2)C(O)C(O)C1O ; CC1OC(OC2C(NC(=O)c3cc(O)nc(O)n3)CC(C(=O)NCCCc3cn(CCCCC(=O)Nc4ccc(S(=O)(=O)O)c5cc(S(=O)(=O)O)cc(S(=O)(=O)O)c45)nn3)CC2OC2OC(CO)C(O)C(O[C@@H](CC3CCCCC3)C(=O)O)C2OC(=O)c2ccccc2)C(O)C(O)C1O] 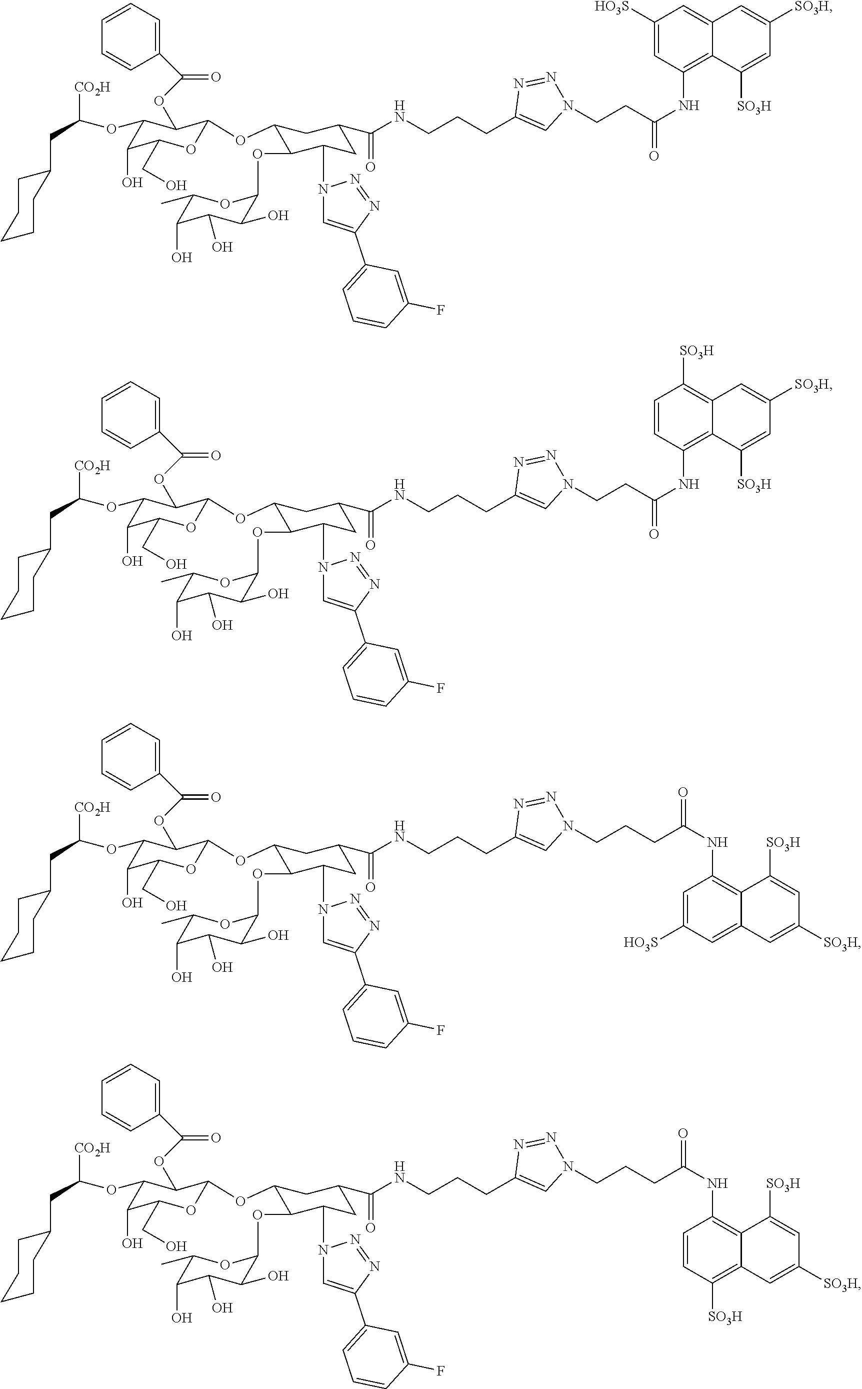 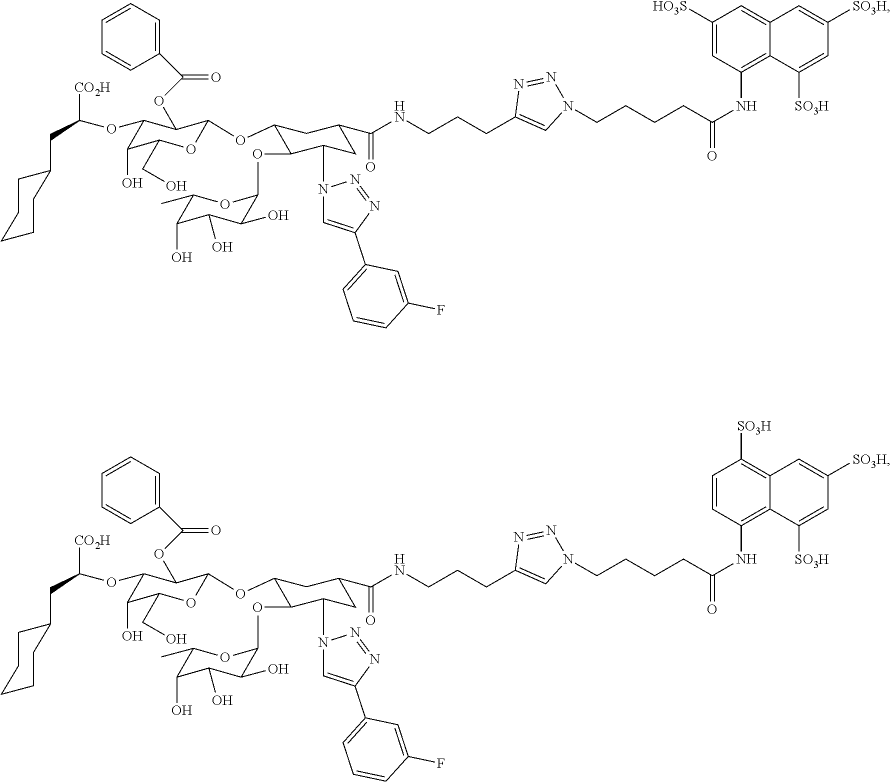 [SMILES: CC1OC(OC2C(OC3OC(CO)C(O)C(O[C@@H](CC4CCCCC4)C(=O)O)C3OC(=O)c3ccccc3)CC(C(=O)NCCCc3cn(CCCCC(=O)Nc4cc(S(=O)(=O)O)cc5cc(S(=O)(=O)O)cc(S(=O)(=O)O)c45)nn3)CC2n2cc(-c3cccc(F)c3)nn2)C(O)C(O)C1O ; CC1OC(OC2C(OC3OC(CO)C(O)C(O[C@@H](CC4CCCCC4)C(=O)O)C3OC(=O)c3ccccc3)CC(C(=O)NCCCc3cn(CCCCC(=O)Nc4ccc(S(=O)(=O)O)c5cc(S(=O)(=O)O)cc(S(=O)(=O)O)c45)nn3)CC2n2cc(-c3cccc(F)c3)nn2)C(O)C(O)C1O]
and pharmaceutically acceptable salts thereof.

23. (canceled)

24. A composition comprising a compound of claim 1 and an additional pharmaceutically acceptable ingredient.

25. A method for modulating a selectin-mediated function comprising administering to a subject in need thereof an effective amount of a compound of claim 1 and optionally an additional pharmaceutically acceptable ingredient.

26. The method of claim 25, wherein the selectin-mediated function is selectin-mediated intercellular adhesion.

27. The method of claim 25, wherein the selectin-mediated function is inhibited.

28. A method for contacting a cell expressing a selectin to modulate the selectin's function comprising administering to a subject in need thereof an effective amount of a compound of claim 1 and optionally an additional pharmaceutically acceptable ingredient.

29. A method for inhibiting the development of a condition associated with an excessive selectin-mediated function comprising administering to a subject in need thereof an effective amount of a compound of claim 1 and optionally an additional pharmaceutically acceptable ingredient.

30. A method for inhibiting rejection of a transplanted tissue comprising administering to a subject in need thereof an effective amount of a compound of claim 1 and optionally an additional pharmaceutically acceptable ingredient.

31. A method for treating sickle cell disease or complications associated therewith comprising administering to a subject in need thereof an effective amount of a compound of claim 1 and optionally an additional pharmaceutically acceptable ingredient.

32. (canceled)

33. (canceled)

34. (canceled)

35. A method of treatment comprising administering to a subject in need thereof an effective amount of a compound of claim 1 and optionally an additional pharmaceutically acceptable ingredient wherein said treatment is selected from the group consisting of treatment for vaso-occlusive crisis, treatment for graft versus host disease or complications associated therewith, treatment for sinusoidal obstruction syndrome or complications associated therewith, treatment for epilepsy, and treatment for cancers of the blood and complications associated therewith.

36. The method of claim 35, wherein said cancers of the blood are selected from the group consisting of acute myelogenous leukemia, acute lymphoblastic leukemia, chronic myelogenous leukemia, and multiple myeloma.

37. (canceled)
